SEQUENCE LISTING (20 sequences)

SEQ ID NO: 1 - DNA, 1737 bp, synthetic construct
  misc_feature 1..1737: note = cDNA of Brassica napus Babyboom
  source 1..1737: mol_type = other DNA
atggataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaat

SEQ ID NO: 2 - protein, 579 aa, Brassica napus
MDNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 3 - DNA, 1974 bp, synthetic construct
  misc_feature 1..1974: note = cDNA sequence of Brassica napus Babyboom with 3'UTR
  source 1..1974: mol_type = other DNA
atggataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattagaaaaaatagttaaagatctttagttatatgcgttgttgtgtgctggtgaacagtgtgatactttgattatgtttttttctttctctttttctttttcttggttaatttcttaagacttatttttagtttccattagttggataaattttcagactaaaaaaaaaaaaaaaaaaaaactcgagggggggcccggtaccgagctcgaatttccccgatcgttcaaacatttgtacc

SEQ ID NO: 4 - DNA, 2603 bp, synthetic construct
  misc_feature 1..2603: note = DNA sequence of Brassica napus Babyboom with rat glucocorticoid receptor domain
  source 1..2603: mol_type = other DNA
atggataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaatggatctacaaagaaaaaaatcaaagggattcagcaagccactgcaggagtctcacaagacacttcggaaaatcctaacaaaacaatagttcctgctgcattaccacagctcacccctaccttggtgtcactgctggaggtgattgaacccgaggtgttgtatgcaggatatgatagctctgttccagattcagcgtggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgagttcccagagatgttagctgaaatcatcactaatcagataccaaaatattcaaatggaaatatcaaaaagctcctgtttcatcaaaaatgacaccc

SEQ ID NO: 5 - DNA, 11632 bp, synthetic construct
  misc_feature 1..11632: note = vector pZFNnptII
  source 1..11632: mol_type = other DNA
cggccgcgtcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgagtcgagtttttcagcaagattctagacttgtacagctcgtccatgccgtacaggaacaggtggtggcggccctcggagcgctcgtactgttccacgatggtgtagtcctcgttgtgggaggtgatgtccagcttggtgtccacgtagtagtagccgggcagttgcacgggcttcttggccatgtagatggtcttgaactccaccaggtagtggccgccgtccttcagcttcagggcctggtggatctcgcccttcagcacgccgtcgcgggggtacaggcgctcggtggaggcctcccagcccatggtcttcttctgcattacggggccgtcgggggggaagttggtgccgcgcatcttcaccttgtagatcagcgtgccgtcctgcagggaggagtcctgggtcacggtcaccagaccgccgtcctcgaagttcatcacgcgctcccacttgaagccctcggggaaggacagcttcttgtaatcggggatgtcggcggggtgcttcacgtacgccttggagccgtacatgaactggggggacaggatgtcccaggcgaagggcagggggccgcccttggtcaccttcagcttggcggtctgggtgccctcgtaggggcggccctcgccctcgccctcgatctcgaactcgtggccgttcatggagccctccatgcgcaccttgaagcgcatgaactctttgatgacctcctcgcccttgctcaccatggtggcgggatcgcgccctatcgttcgtaaatggtgaaaattttcagaaaattgcttttgctttaaaagaaatgatttaaattgctgcaatagaagtagaatgcttgattgcttgagattcgtttgttttgtatatgttgtgttgagaggatccactagttctagagcggccgccaccgcggtggagctcgaattctcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacctgcaggcatgcaagctctagtaaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggtccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatgg

SEQ ID NO: 6 - DNA, 801 bp, synthetic construct
  misc_feature 1..801: note = Neomycin phosphotransferase
  source 1..801: mol_type = other DNA
atgggagcttggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaacttcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgactcatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttc

SEQ ID NO: 7 - DNA, 702 bp, synthetic construct
  misc_feature 1..702: note = tdTomato, orange fluorescent protein
  source 1..702: mol_type = other DNA
atggtgagcaagggcgaggaggtcatcaaagagttcatgcgcttcaaggtgcgcatggagggctccatgaacggccacgagttcgagatcgagggcgagggcgagggccgcccctacgagggcacccagaccgccaagctgaaggtgaccaagggcggccccctgcccttcgcctgggacatcctgtccccccagttcatgtacggctccaaggcgtacgtgaagcaccccgccgacatccccgattacaagaagctgtccttccccgagggcttcaagtgggagcgcgtgatgaacttcgaggacggcggtctggtgaccgtgacccaggactcctccctgcaggacggcacgctgatctacaaggtgaagatgcgcggcaccaacttcccccccgacggccccgtaatgcagaagaagaccatgggctgggaggcctccaccgagcgcctgtacccccgcgacggcgtgctgaagggcgagatccaccaggccctgaagctgaaggacggcggccactacctggtggagttcaagaccatctacatggccaagaagcccgtgcaactgcccggctactactacgtggacaccaagctggacatcacctcccacaacgaggactacaccatcgtggaacagtacgagcgctccgagggccgccaccacctgttcctgtacggcatggacgagctgtacaag

SEQ ID NO: 8 - DNA, 691 bp, synthetic construct
  misc_feature 1..691: note = double 35s promoter
  source 1..691: mol_type = other DNA
gttcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatacatggtggagcacgacactctcgtctactccaagaatatcaaagatacagtctcagaagaccagagggctattgagacttttcaacaaagggtaatatcgggaaacctcctcggattccattgcccagctatctgtcacttcatcgaaaggacagtagaaaaggaagatggcttctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagg

SEQ ID NO: 9 - DNA, 698 bp, synthetic construct
  misc_feature 1..698: note = Col E1 origin of replication
  source 1..698: mol_type = other DNA
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaa

SEQ ID NO: 10 - DNA, 3706 bp, synthetic construct
  misc_feature 1..3706: note = pVS1 Replicon
  source 1..3706: mol_type = other DNA
cgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcacc

SEQ ID NO: 11 - DNA, 1008 bp, Escherichia coli
  source 1..1008: mol_type = unassigned DNA
atgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaa

SEQ ID NO: 12 - DNA, 909 bp, synthetic construct
  misc_feature 1..909: note = cDNA sequence of ZmWUS2
  source 1..909: mol_type = other DNA
atggcggccaatgcgggcggcggtggagcgggaggaggcagcggcagcggcagcgtggctgcgccggcggtgtgccgccccagcggctcgcggtggacgccgacgccggagcagatcaggatgctgaaggagctctactacggctgcggcatccggtcgcccagctcggagcagatccagcgcatcaccgccatgctgcggcagcacggcaagatcgagggcaagaacgtcttctactggttccagaaccacaaggcccgcgagcgccagaagcgccgcctcaccagcctcgacgtcaacgtgcccgccgccggcgcggccgacgccaccaccagccaactcggcgtcctctcgctgtcgtcgccgccgccttcaggcgcggcgcctccctcgcccaccctcggcttctacgccgccggcaatggcggcggatcggctgtgctgctggacacgagttccgactggggcagcagcggcgctgccatggccaccgagacatgcttcctccaggactacatgggcgtgacggacacgggcagctcgtcgcagtggccacgcttctcgtcgtcggacacgataatggcggcggccgcggcgcgggcggcgacgacgcgggcgcccgagacgctccctctcttcccgacctgcggcgacgacggcggcagcggtagcagcagctacttgccgttctggggtgccgcgtccacaactgccggcgccacttcttccgttgcgatccagcagcaacaccagctgcaggagcagtacagcttttacagcaacagcaacagcacccagctggccggcaccggcaaccaagacgtatcggcaacagcagcagcagccgccgccctggagctgagcctcagctcatggtgctccccttaccctgctgcagggagtatgtga

SEQ ID NO: 13 - protein, 302 aa, Zea mays
MAANAGGGGAGGGSGSGSVAAPAVCRPSGSRWTPTPEQIRMLKELYYGCGIRSPSSEQIQRITAMLRQHGKIEGKNVFYWFQNHKARERQKRRLTSLDVNVPAAGAADATTSQLGVLSLSSPPPSGAAPPSPTLGFYAAGNGGGSAVLLDTSSDWGSSGAAMATETCFLQDYMGVTDTGSSSQWPRFSSSDTIMAAAAARAATTRAPETLPLFPTCGDDGGSGSSSYLPFWGAASTTAGATSSVAIQQQHQLQEQYSFYSNSNSTQLAGTGNQDVSATAAAAAALELSLSSWCSPYPAAGSM

SEQ ID NO: 14 - DNA, 13362 bp, synthetic construct
  misc_feature 1..13362: note = IPR232-pS-01
  source 1..13362: mol_type = other DNA
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccggaagacaaagatagcccggggcctgcaggaccaatgcattggatctggagacccgccggcggcagaagagatccaacaaaacattcacaatggattatagaaacatttgtttattcattataatgagatcttacattcatttaatattagaaaaagccacaaattcataacacaacaagccaagaaaaaaacacaaacttaagcacacaagctttttatttgacacaccaaatatttcatcttcatcttcatatgagctcgtcgacggtaccccctcaattagttacatcaccactttgtacaagaaagctgggtgtcatttttgatgaaacaggagctttttgatatttccatttgaatattttggtatctgattagtgatgatttcagctaacatctctgggaactcaatactcatggtcttatccaaaaatgtctggaagcagtaggtaaggagattctcaaccacctcatgcatggagtccagaagctttgtcagttggtaaaaccgttgccagttctgactggagttcccttcccttttgacgatggcttttcctagctctttgatataagtcattcgaatctcatcaaataactcttggctcttcagaccttccttaggaactgaggagagaagcagtaaggttttcatacagagatactcttcataggatacctgcaatctttgtaattcagaggagacaaacagcatgtgtttacattggtcatacatgcagggtagagacattctctgctcattaataatcagatcaggagcaaagcagagcaggtttccacttgattgtctgtatgatctccaacccagggcaaatgccatgagaaacatccatgagtactgtagcagggtcatttggtcatccaggtgtaagtttctgaagcctggtatcgcctttgcccatttcactgctgcaatcacttgacgcccacctaacatgttgagtgtggtcataattctccacgctgaatctggaacagagctatcatatcctgcatacaacacctcgggttcaatcacctccagcagtgacaccaaggtaggggtgagctgtggtaatgcagcaggaactattgttttgttaggattttccgaagtgtcttgtgagactcctgcagtggcttgctgaatccctttgatttttttctttgtagatccattgtcgttccaaactgtaaatgttggagcaacttctccaccaccttccccatggtaatacatattagagccaacattattcgtcattgccgcgggaaaatctccacctggcgactgctgggtctgctgctgctgagcaaagtaataatggtttcttgcgttataatcaaactcactagcagcgtaggcatcgcagttaaccggggctgcaaatccttgataaccaccataaccaacaacatttccacaaacagtaaccgaatcatccgaaacagaactttgatgatcgatattagtcatgaggctctgcgtgttgctcaagaaatggtgttgatcatcctcttgtttgaaacaagccctagcactctccgaagacaagtttcctccattgtaataataaccattgtacctctcttgatgttggtgcaataagctcaaatctactccctgatgatgctgaaccgcagcgttttgccaaccgctagcactattctcactctctgaaacgttattactgatcatcatcatacttggaaccggacggttagcctccttgagacgttttgcggcgctaccaataggaagactagggctttcgaggattgctttaacgttgtatctgttcatgtcgaagttagtcactgcggttaatcctctgaatttgatggccgcaatgtcgtatgcctctgcagcttcttcttgtgtgccaaaagttcccaagtagaggtctttgttaccggcgactcttcctatcctagcttgccatcttccatgttggtgatgtcttgttactccacgataaatcgatgcaccacgagagaaaccactacttttcctgcgcagtgaggcaacatactcttgccttgtcatgtgcttcatctcttctacctctttttcatattcgctcatggggaagttagtagtagtggtggttccccaatacttgagtgcggctaaatcataagccctagctgctttttcttctttgtcataacctcccaaataaacttgtcttcctttgcgcgtttggccttctcttttacaactattatcccataaatgtgcctcatatcttcctgtccaccgatgccttgtaacaccgcggtatatagacgtcctctgtccaaaactctcaatagttttcttcggtgtagcttcaacgctatcatcaatagtcttcccttgggcaacaacgttgttattgctgtcgttgttgttatcacaagaagtagatgagttcattgagagggacaggccttttgcagcattgccattttcttgattatcaacattatccacgggttgatttctcagccatgtctttatcatcgaaagtcctagtgagccaccaccaccgtctcctcctccataacagccacttccacttccatctccaacgttttcgttggtgttgtaaatcgtggtggtgcggccaaggaaattctcaagctttggtccatcttgctcatcgttgtggatgttattgcatgcacaaccattgatgtcccaatctcgggagtgactattgttgtctctggtgaaagcatcgacgacgacaccaaagggagaaggaaacgatgtttggatggctgaagactcatcggaggcagcggtcggatcgtaacagtactctccggcgacatctacgacggttgtggtggtggaagagtagacgtccttacgatggtgattttgttcataaggagagagagaaaagcctaaccagttattatccattgagcctgcttttttgtacaaacttgtgatcgggatctgtaattgtaatgttgtttgttgtttgttgttgttggtaattgtggatcctctagagtcccccgtgttctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgcctcgccggcgggtctcgggatcccctgcaggactagtggatttgtcttcaaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcacatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgaaggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 15 - DNA, 12734 bp, synthetic construct
  misc_feature 1..12734: note = IPR252-pS-01
  source 1..12734: mol_type = other DNA
ccggattgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggtcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgagtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgaagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccattttctgtgattttggatgtgtttttgtatggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcattccggactgtggggccatatcccagaactggttgagtcggtccaacacctgggtgccaatcatgtcgatggtggggtatggccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagaggccatggcggccggaagacaaagatagcccggggcctgcaggaccaatgcattggatctggagacccgccggcggcagaagagatccaacaaaacattcacaatggattatagaaacatttgtttattcattataatgagatcttacattcatttaatattagaaaaagccacaaattcataacacaacaagccaagaaaaaaacacaaacttaagcacacaagctttttatttgacacaccaaatatttcatcttcatcttcatatgagctcgtcgacggtaccccctcaattagttacatcaccactttgtacaagaaagctgggtacaaatgtttgaacgatcggggaaattcgagctcggtaccgggccccccctcgagtttttttttttttttttttttagtctgaaaatttatccaactaatggaaactaaaaataagtcttaagaaattaaccaagaaaaagaaaaagagaaagaaaaaaacataatcaaagtatcacactgttcaccagcacacaacaacgcatataactaaagatctttaactattttttctaattgtcgttccaaactgtaaatgttggagcaacttctccaccaccttccccatggtaatacatattagagccaacattattcgtcattgccgcgggaaaatctccacctggcgactgctgggtctgctgctgctgagcaaagtaataatggtttcttgcgttataatcaaactcactagcagcgtaggcatcgcagttaaccggggctgcaaatccttgataaccaccataaccaacaacatttccacaaacagtaaccgaatcatccgaaacagaactttgatgatcgatattagtcatgaggctctgcgtgttgctcaagaaatggtgttgatcatcctcttgtttgaaacaagccctagcactctccgaagacaagtttcctccattgtaataataaccattgtacctctcttgatgttggtgcaataagctcaaatctactccctgatgatgctgaaccgcagcgttttgccaaccgctagcactattctcactctctgaaacgttattactgatcatcatcatacttggaaccggacggttagcctccttgagacgttttgcggcgctaccaataggaagactagggctttcgaggattgctttaacgttgtatctgttcatgtcgaagttagtcactgcggttaatcctctgaatttgatggccgcaatgtcgtatgcctctgcagcttcttcttgtgtgccaaaagttcccaagtagaggtctttgttaccggcgactcttcctatcctagcttgccatcttccatgttggtgatgtcttgttactccacgataaatcgatgcaccacgagagaaaccactacttttcctgcgcagtgaggcaacatactcttgccttgtcatgtgcttcatctcttctacctctttttcatattcgctcatggggaagttagtagtagtggtggttccccaatacttgagtgcggctaaatcataagccctagctgctttttcttctttgtcataacctcccaaataaacttgtcttcctttgcgcgtttggccttctcttttacaactattatcccataaatgtgcctcatatcttcctgtccaccgatgccttgtaacaccgcggtatatagacgtcctctgtccaaaactctcaatagttttcttcggtgtagcttcaacgctatcatcaatagtcttcccttgggcaacaacgttgttattgctgtcgttgttgttatcacaagaagtagatgagttcattgagagggacaggccttttgcagcattgccattttcttgattatcaacattatccacgggttgatttctcagccatgtctttatcatcgaaagtcctagtgagccaccaccaccgtctcctcctccataacagccacttccacttccatctccaacgttttcgttggtgttgtaaatcgtggtggtgcggccaaggaaattctcaagctttggtccatcttgctcatcgttgtggatgttattgcatgcacaaccattgatgtcccaatctcgggagtgactattgttgtctctggtgaaagcatcgacgacgacaccaaagggagaaggaaacgatgtttggatggctgaagactcatcggaggcagcggtcggatcgtaacagtactctccggcgacatctacgacggttgtggtggtggaagagtagacgtccttacgatggtgattttgttcataaggagagagagaaaagcctaaccagttattatccattgagcctgcttttttgtacaaacttgtgatcgggatctgtaattgtaatgttgtttgttgtttgttgttgttggtaattgtggatcctctagagtcccccgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcaattgccctttggtcttctgagactgtatctttgatatttttggagtagacaagtgtgtcgtgctccaccatgttgacgaagattttcttcttgtcattgagtcgtaagagactctgtatgaactgttcgccagtctttacggcgagttctgttaggtcctctatttgaatctttgactccatggcctttgattcagtgggaactacctttttagagactccaatctctattacttgcctcgccggcgggtctcgggatcccctgcaggactagtggatttgtcttcaaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccgaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcacatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgaaggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagttcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgtttctatgcttcaattctgctctatagatgttcttctagtcttaacttttagaaggcacaacatttgaaatgttggtaatttttctgctgggtagtgaaccattgtaatttgtgtttgcttatattatccgttgggtttctctatgttatctgtggggccatatcccagaactggttgagtcggtccaacacctgggtgccaatcatgtcgatggtggggtatggt

SEQ ID NO: 16 - DNA, 5472 bp, synthetic construct
  misc_feature 1..5472: note = IPR 252-pK-02
  source 1..5472: mol_type = other DNA
cggacgaccctgcgcgctcagacgaaacaccgccgcatcagagcagccaatggtctgctgcgcccaatcatagccaaacagacgttccacccacgctgccgggctacccgcatgcaggccatcctgttcaatcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggactagaggcccttaaggccttgaagacaaatccactagtcctgcaggggatcccgagacccgccggcgaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgggggactctagaggatccacaattaccaacaacaacaaacaacaaacaacattacaattacagatcccgatcacaagtttgtacaaaaaagcaggctcaatggataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattagaaaaaatagttaaagatctttagttatatgcgttgttgtgtgctggtgaacagtgtgatactttgattatgtttttttctttctctttttctttttcttggttaatttcttaagacttatttttagtttccattagttggataaattttcagactaaaaaaaaaaaaaaaaaaaaactcgagggggggcccggtaccgagctcgaatttccccgatcgttcaaacatttgtacccagctttcttgtacaaagtggtgatgtaactaattgagggggtaccgtcgacgagctcatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgccgccggcgggtctccagatccaatgcattggtcctgcaggccccgggctatctttgtcttccggccgccatggccagatcgtacccaattcgccctatagtgagtcgtattacaattcactggccgtcgttttacaacgtcgtgactgggaaaaccctggcgttacccaacttaatcgccttgcagcacatccccctttcgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaattcatccagcagacgataaaacgcaatacgctggctatccggtgccgcaatgccatacagcaccagaaaacgatccgcccattcgccgcccagttcttccgcaatatcacgggtggccagcgcaatatcctgataacgatccgccacgcccagacggccgcaatcaataaagccgctaaaacggccattttccaccataatgttcggcaggcacgcatcaccatgggtcaccaccagatcttcgccatccggcatgctcgctttcagacgcgcaaacagctctgccggtgccaggccctgatgttcttcatccagatcatcctgatccaccaggcccgcttccatacgggtacgcgcacgttcaatacgatgtttcgcctgatgatcaaacggacaggtcgccgggtccagggtatgcagacgacgcatggcatccgccataatgctcactttttctgccggcgccagatggctagacagcagatcctgacccggcacttcgcccagcagcagccaatcacggcccgcttcggtcaccacatccagcaccgccgcacacggaacaccggtggtggccagccagctcagacgcgccgcttcatcctgcagctcgttcagcgcaccgctcagatcggttttcacaaacagcac

SEQ ID NO: 17 - DNA, 1260 bp, synthetic construct
  misc_feature 1..1260: note = cDNA BnBBM Version with shortend N terminus
  source 1..1260: mol_type = other DNA
atgctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagcgccgcaaaacgtctcaaggaggctaaccgtccggttccaagtatgatgatgatcagtaataacgtttcagagagtgagaatagtgctagcggttggcaaaacgctgcggttcagcatcatcagggagtagatttgagcttattgcaccaacatcaagagaggtacaatggttattattacaatggaggaaacttgtcttcggagagtgctagggcttgtttcaaacaagaggatgatcaacaccatttcttgagcaacacgcagagcctcatgactaatatcgatcatcaaagttctgtttcggatgattcggttactgtttgtggaaatgttgttggttatggtggttatcaaggatttgcagccccggttaactgcgatgcctacgctgctagtgagtttgattataacgcaagaaaccattattactttgctcagcagcagcagacccagcagtcgccaggtggagattttcccgcggcaatgacgaataatgttggctctaatatgtattaccatggggaaggtggtggagaagttgctccaacatttacagtttggaacgacaattag

SEQ ID NO: 18 - DNA, 1170 bp, synthetic construct
  misc_feature 1..1170: note = cDNA BnBBM Version with shortend C terminus
  source 1..1170: mol_type = other DNA
atggataataactggttaggcttttctctctctccttatgaacaaaatcaccatcgtaaggacgtctactcttccaccaccacaaccgtcgtagatgtcgccggagagtactgttacgatccgaccgctgcctccgatgagtcttcagccatccaaacatcgtttccttctccctttggtgtcgtcgtcgatgctttcaccagagacaacaatagtcactcccgagattgggacatcaatggttgtgcatgcaataacatccacaacgatgagcaagatggaccaaagcttgagaatttccttggccgcaccaccacgatttacaacaccaacgaaaacgttggagatggaagtggaagtggctgttatggaggaggagacggtggtggtggctcactaggactttcgatgataaagacatggctgagaaatcaacccgtggataatgttgataatcaagaaaatggcaatgctgcaaaaggcctgtccctctcaatgaactcatctacttcttgtgataacaacaacgacagcaataacaacgttgttgcccaagggaagactattgatgatagcgttgaagctacaccgaagaaaactattgagagttttggacagaggacgtctatataccgcggtgttacaaggcatcggtggacaggaagatatgaggcacatttatgggataatagttgtaaaagagaaggccaaacgcgcaaaggaagacaagtttatttgggaggttatgacaaagaagaaaaagcagctagggcttatgatttagccgcactcaagtattggggaaccaccactactactaacttccccatgagcgaatatgaaaaagaggtagaagagatgaagcacatgacaaggcaagagtatgttgcctcactgcgcaggaaaagtagtggtttctctcgtggtgcatcgatttatcgtggagtaacaagacatcaccaacatggaagatggcaagctaggataggaagagtcgccggtaacaaagacctctacttgggaacttttggcacacaagaagaagctgcagaggcatacgacattgcggccatcaaattcagaggattaaccgcagtgactaacttcgacatgaacagatacaacgttaaagcaatcctcgaaagccctagtcttcctattggtagctag

SEQ ID NO: 19 - protein, 418 aa, synthetic construct
  REGION 1..418: note = PRT BnBBM Version with shortend N terminus
LSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGSAAKRLKEANRPVPSMMMISNNVSESENSASGWQNAAVQHHQGVDLSLLHQHQERYNGYYYNGGNLSSESARACFKQEDDQHHFLSNTQSLMTNIDHQSSVSDDSVTVCGNVVGYGGYQGFAAPVNCDAYAASEFDYNARNHYYFAQQQQTQQSPGGDFPAAMTNNVGSNMYYHGEGGGEVAPTFTVWNDN

SEQ ID NO: 20 - protein, 389 aa, synthetic construct
  REGION 1..389: note = PRT BnBBM Version with shortend C terminus
MDNNWLGFSLSPYEQNHHRKDVYSSTTTTVVDVAGEYCYDPTAASDESSAIQTSFPSPFGVVVDAFTRDNNSHSRDWDINGCACNNIHNDEQDGPKLENFLGRTTTIYNTNENVGDGSGSGCYGGGDGGGGSLGLSMIKTWLRNQPVDNVDNQENGNAAKGLSLSMNSSTSCDNNNDSNNNVVAQGKTIDDSVEATPKKTIESFGQRTSIYRGVTRHRWTGRYEAHLWDNSCKREGQTRKGRQVYLGGYDKEEKAARAYDLAALKYWGTTTTTNFPMSEYEKEVEEMKHMTRQEYVASLRRKSSGFSRGASIYRGVTRHHQHGRWQARIGRVAGNKDLYLGTFGTQEEAAEAYDIAAIKFRGLTAVTNFDMNRYNVKAILESPSLPIGS

(19) United States
(12) Patent Application Publication
(10) Pub. No.: US 2026/0240100 A1
(43) Pub. Date: Aug. 20, 2026

(54) PROMOTING REGENERATION AND TRANSFORMATION IN PLANTS

(71) Applicants: KWS SAAT SE & Co. KGaA, Einbeck (DE); STICHTING WAGENINGEN RESEARCH, Wageningen (NL)

(72) Inventors: Jixiang KONG, Einbeck (DE); Susana MARTIN-ORTIGOSA, Einbeck (DE); David PACHECO VILLALOBOS, Einbeck (DE); Christine KASTNER, Einbeck (DE); Frank LUDEWIG, Einbeck (DE); Anneke HORSTMAN, Wageningen (NL); Kimberly Anne BOUTILIER, Wageningen (NL)

(73) Assignees: KWS SAAT SE & Co. KGaA, Einbeck (DE); STICHTING WAGENINGEN RESEARCH, Wageningen (NL)

(21) Appl. No.: 19/450,453

(22) Filed: Jan. 15, 2026

Related U.S. Application Data
(63) Continuation of application No. 18/268,856, filed on Dec. 22, 2021, now Pat. No. 12,527,270.

(30) Foreign Application Priority Data
Dec. 22, 2020 (EP) 20216530.4

(51) Int. Cl. A01H 4/00; C12N 15/82

(52) U.S. Cl. CPC A01H 4/008; C12N 15/8205

(57) ABSTRACT

The present invention relates to the field of plant breeding and in particular to the regeneration of plants from cells and other tissues. More particularly, the invention provides methods and means for improving callus and shoot formation and regeneration of plants using sodium nitroprusside (SNP) in combination with Brassica napus BABYBOOM (BnBBM).

### CROSS-REFERENCE TO RELATED APPLICATIONS

This application is a continuation of U.S. application Ser. No. 18/268,856, filed Jun. 21, 2023, which is a U.S. National Phase of International Patent Application No. PCT/EP2021/087315, filed on Dec. 22, 2021, which claims priority to European Application No. 20216530.4 filed Dec. 22, 2020. The entire contents of these applications are incorporated herein by reference in their entirety.

### SEQUENCE LISTING

The instant application contains a Sequence Listing which has been submitted electronically in XML file format and is hereby incorporated by reference in its entirety. Said XML copy, created on Jan. 5, 2026, is named 245761_000267_SL.xml and is 86,967 bytes in size.

### TECHNICAL FIELD

The present invention relates to the technical field of plant regeneration and induction of somatic embryogenesis, preferably in sugar beet and corn. More particularly, the invention provides methods and means for improving callus and shoot formation and regeneration of plants using sodium nitroprusside (SNP).

### TECHNICAL BACKGROUND

The ability to regenerate whole plants via tissue culture is a well-described and much applied phenomenon. The most commonly used methods for plant regeneration from somatic cells involve culturing explants on medium containing growth regulators to induce callus formation, followed by organogenesis or embryogenesis from the dedifferentiated callus. Dissecting the phenomenon of in vitro regeneration at the molecular-genetic level has been difficult due to the length of the regeneration process and the many developmental events that take place as explants dedifferentiate and then redifferentiate new tissues and organs. Mutant screens and functional studies have been particularly helpful in identifying proteins that control developmental pathways.

Among the proteins controlling regeneration are those that induce somatic embryogenesis from competent somatic cells. The BABY BOOM (BBM) AP2/ERF domain protein is a seed and root-meristem expressed transcription factor that was identified as marker for embryo development in Brassica napus microspore-derived embryo cultures (Boutilier, Kim, et al. (“Ectopic expression of BABY BOOM triggers a conversion from vegetative to embryonic growth.” The Plant Cell 14.8 (2002): 1737-1749.) The authors describe that BABY BOOM (BBM) showing similarity to the AP2/ERF family of transcription factors and expressed preferentially in developing embryos and seeds is involved in in vitro induction of embryo development from immature pollen grains of Brassica napus (microspore embryogenesis). Ectopic expression of BBM in Arabidopsis and Brassica led to the spontaneous formation of somatic embryos and cotyledon-like structures on transgenic seedlings. Ectopic BBM expression induced additional pleiotropic phenotypes, including neoplastic growth and alterations in leaf and flower morphology.

Florez, Sergio L., et al. (“Enhanced somatic embryogenesis in Theobroma cacao using the homologous BABY BOOM transcription factor.” BMC Plant Biology 15.1 (2015): 121.) describe the use of the transcription factor Baby Boom (BBM) derived from Theobroma cacao to promote the transition of somatic cacao cells from the vegetative to embryonic state. While transient ectopic expression of TcBBM provided only moderate enhancements in embryogenic potential, constitutive overexpression dramatically increased somatic embryogenesis proliferation but also appeared to inhibit subsequent development.

Srinivasan, Chinnathambi, et al. (“Heterologous expression of the BABY BOOM AP2/ERF transcription factor enhances the regeneration capacity of tobacco (Nicotiana tabacum L.).” Planta 225.2 (2007): 341.) examined the effect of ectopic BBM expression on the development and regenerative capacity of tobacco (Nicotiana tabacum L.) through heterologous expression of Arabidopsis and B. napus BBM genes. 35S::BBM tobacco lines exhibited a number of phenotypes including callus formation, leaf rumpling, and sterility, but they did not undergo spontaneous somatic embryogenesis. 35S::BBM plants with severe ectopic expression phenotypes could not be assessed for enhanced regeneration at the seedling stage due to complete male and female sterility of the primary transformants. Therefore fertile BBM ectopic expression lines with strong misexpression phenotypes were generated by expressing a steroid-inducible, post-translationally controlled BBM fusion protein (BBM:GR) under the control of a 35S promoter. These lines exhibited spontaneous shoot and root formation upon the application of DEX, while somatic embryogenesis could be induced from in vitro germinated seedling hypocotyls cultured on media supplemented with cytokinin.

WO2011/003850A1 describes a general method for providing fertile plants via induction of BBM during transformation. Recovery of fertile plants however requires phytohormones like auxins or cytokinins.

Sodium nitroprusside (SNP) is a well-known medication used for example to lower blood pressure. However, in plant biotechnology SNP was also described for plant regeneration. Xu et al. (Effects of sodium nitroprusside on callus induction and shoot regeneration in micropropagated Dioscorea opposite; Plant Growth Regulation volume 59, Article number: 279 (2009)) described the effects of sodium nitroprusside on Dioscorea opposite callus induction and shoot regeneration. Chin Tan et al. (Effects of sodium nitroprusside on shoot multiplication and regeneration of Vanilla planifolia Andrews. In Vitro Cellular & Developmental Biology—Plant volume 49, pages 626-630 (2013)) described effects of a NO donor like sodium nitroprusside (SNP), on shoot multiplication and regeneration of Vanilla planifolia. Charu Kalra and Shashi B. Babbar (Nitric oxide promotes in vitro organogenesis in Linum usitatissimum L. Plant Cell, Tissue and Organ Culture (PCTOC) volume 103, pages 353-359(2010)) described the effects of nitric oxide (NO) donors like SNP on caulogenesis, shoot organogenesis and rhizogenesis from hypocotyl explants of Linum usitatissimum. Han et al., (Sodium nitroprusside promotes multiplication and regeneration of Malus hupehensis in vitro plantlets. Plant Cell, Tissue and Organ Culture volume 96, pages 29-34 (2009)) described the effects of sodium nitroprusside (SNP) on the multiplication, regeneration and rooting of Malus hupehensis in tissue culture. Sarropoulou et al., (In vitro plant regeneration from leaf explants of the cherry rootstocks CAB-6P, Gisela 6, and MxM 14 using sodium nitroprusside. In Vitro Cellular & Developmental Biology—Plant volume 50, pages 226-234 (2014)) described multiplication of Prunus cerasus L. rootstocks using tissue culture approaches including SNP which lead to promoted callus induction, in vitro shoot proliferation, and rooting from leaf explants. U.S. Pat. No. 7,855,325 & U.S. Pat. No. 7,388,126 describe SNP as media component in agrobacterium-based corn transformation experiments to increase transformation efficiency.

However, the use of SNP in callus regeneration in sugar beet as well as the use of SNP in combination with BBM-mediated shoot induction has never been described. So far, induction of somatic embryogenesis through BBM transformation has only been described for Capsicum annuum, Nicotiana tabacum, Arabidopsis thaliana, Brassica napus, Theobroma cacao, Populus tomentosa, but never for Beta vulgaris which belongs to one of the economically most relevant crops. Somatic embryogenesis based transformation was not possible before in Beta vulgaris and has a positive impact on the overall timeline for biotech processes in sugar beet.

### SUMMARY OF THE INVENTION

In the present invention it was found, that by applying the chemical SNP to plant cells the induction of callus even from recalcitrant genotypes was significantly promoted. SNP was found to have a beneficial effect on the complex mechanism of callus induction, especially for recalcitrant genotypes of sugar beet, corn and spinach. The resulting callus could be regenerated into normal shoots and the produced plants show a normal phenotype. Best results for shoot induction of recalcitrant genotypes were obtained when the effects of SNP and Brassica napus BBM (BnBBM) were consecutively combined. BnBBM is capable to promote somatic embryogenesis of Beta vulgaris, Zea mays (corn) and Spinacia oleracea (spinach). Somatic embryogenesis is achieved in a hormone independent way resulting in fertile plants.

The invention describes nucleotides and methods for the induction of somatic embryogenesis and plant regeneration in e.g. Beta vulgaris, Zea mays and Spinacia oleracea. The present invention leads to fertile plants without pleiotropic phenotypes, enabling transformation of recalcitrant genotypes, reduction of somaclonal variation and a high frequency of co-transformation. In general, the time needed for plant regeneration is significantly shortened with this method and the quality of the obtained plants is also clearly improved in comparison to other regeneration methods. The high quality of the obtained plants can be of specific value for root features e.g. weight, sucrose content as well as for leaf parameters like photosynthesis.

In particular, the invention is about a genotype independent callus-based regeneration method by using inducible or constitutive expression of BBM in combination with SNP resulting in fertile plants. The transformation rate is dramatically increased. The regeneration of plants is hormone-independent, supporting somatic embryogenesis and avoiding organogenesis (e.g. shoot formation without roots). The time needed for transformation is significantly shortened with this invention.

Thus, according to a first aspect, the invention provides a method of promoting somatic embryogenesis or organogenesis comprising the following steps:
(a) inducing callus formation from at least one plant cell comprising incubating the at least one plant cell in the presence of sodium nitroprusside (SNP), in particular in a medium comprising SNP, and
(b) introducing into the at least one plant cell to be used in step (a) or into the at least one cell of the callus obtained in step (a) an expression cassette comprising a coding nucleotide sequence selected from
   (i) a nucleotide sequence of SEQ ID NO: 1, or a sequence which is at least 80% identical to the sequence of SEQ ID NO: 1; and
   (ii) a nucleotide sequence encoding a polypeptide having the amino acid sequence of SEQ ID NO: 2, or a sequence which is at least 80% identical to the sequence of SEQ ID NO: 2,
   wherein the nucleotide sequence is operably linked to a heterologous constitutive regulatory element or a heterologous inducible regulatory element; and
(c) cultivating the callus obtained in step (b) under conditions promoting growth of embryos and/or shoots out of the callus, wherein in the callus the polypeptide is expressed from the expression cassette constitutively or upon induction of the heterologous inducible expression system.

Plant cells suitable for inducing callus in step (a) include embryonic plant cells and somatic plant cells. The plural “plant cells” as used herein must not be understood in that a minimum number of plant cells would be required. In principle, only one plant cell may be sufficient. The way in which these plant cells are provided is not important for the method according to the present invention. Plant cells can be used either in isolated form or as part of a plant tissue. For example, embryonic or somatic plant cells can be provided from an explant isolated from a plant. Either the cells are isolated from the explant or the explant is directly used for the induction of callus tissue.

Which part of a plant is eligible for obtaining an explant depends on the particular plant species. Generally, suitable plant cells can be obtained from hypocotyl, shoot, leaves, buds, flowers and roots of a plant. Preferably, an explant or a part thereof isolated from a plant is used in the method of the invention.

In principle, the present invention is applicable to any plant species, whether monocot or dicot, and even to recalcitrant genotypes. For example, plants which may be subject to the methods and uses of the present invention, can be selected from the group consisting of Hordeum vulgare, Hordeum bulbusom, Sorghum bicolor, Saccharum officinarium, Zea spp., including Zea mays. Setaria italica, Oryza minuta, Oryza sativa, Oryza australiensis, Oryza alta, Triticum aestivum, Triticum durum, Secale cereale, Triticale, Malus domestica, Brachypodium distachyon, Hordeum marinum, Aegilops tauschii, Daucus glochidiatus, Beta spp., including Beta vulgaris, Daucus pusillus, Daucus muricatus, Daucus carota, Eucalyptus grandis, Nicotiana sylvestris, Nicotiana tomentosiformis, Nicotiana tabacum, Nicotiana benthamiana, Solanum lycopersicum, Solanum tuberosum, Coffea canephora, Vitis vinifera, Erythrante guttata, Genlisea aurea, Cucumis sativus, Marus notabilis, Arabidopsis arenosa, Arabidopsis lyrata, Arabidopsis thaliana, Crucihimalaya himalaica, Crucihimalaya wallichii, Cardamine nexuosa, Lepidium virginicum, Capsella bursa pastoris, Olmarabidopsis pumila, Arabis hirsute, Brassica napus, Brassica oleracea, Brassica rapa, Raphanus sativus, Brassica juncacea, Brassica nigra, Eruca vesicaria subsp. sativa, Citrus sinensis, Jatropha curcas, Populus trichocarpa, Medicago truncatula, Cicer yamashitae, Cicer bijugum, Cicer arietinum, Cicer reticulatum, Cicer judaicum, Cajanus cajanifolius, Cajanus scarabaeoides, Phaseolus vulgaris, Glycine max, Gossypium sp., Astragalus sinicus, Lotus japonicas, Torenia fournieri, Allium cepa, Allium fistulosum, Allium sativum, Helianthus annuus, Helianthus tuberosus and/or Allium tuberosum. Particularly preferred are Beta vulgaris, Zea mays, and Spinacia oleracea.

For inducing callus formation, the plant cells are incubated in the presence of SNP. In particular, they are cultivated in a medium containing SNP. With SNP as a media component, callus induction is possible even for recalcitrant genotypes. The concentration of SNP in the medium can range from about 10 pM up to about 1 mM. In order to achieve the desired boost of callus formation, the concentration of SNP in the medium is preferably in a range of 10 pM to 100 pM, for example 25 μM to 75 pM. In addition, further additives for the induction of callus tissue can be added which are well known in the art. In principle, any culture medium known in the art can be supplemented with SNP, in particular a medium commonly used for inducing callus formation. Depending on the plant in question, the composition of the medium may vary. In principle, several types of basal salt mixtures can be added to the medium, but preferably, the medium comprises modified Murashige and Skoog (MS) medium, White's medium, or woody plant medium, most preferably MS medium. Previous studies indicate that callus induction is facilitated in the presence of appropriate amounts and concentrations of auxins and cytokinins alone or in combination with each other in MS medium. According to the invention, these components can also be added preferentially to the culture medium. Exemplary auxins include naphthalene acetic acid (NAA), indole-3-acetic acid (IAA) and indole-3-butyric acid (IBA). Exemplary cytokinins include 6-Benzylaminopurine (BAP) and 6-furfurylamino-purine (kinetin).

Incubation in the medium containing SNP is preferably carried out for about 5-10 weeks, more preferably about 6-8 weeks. Short incubation terms in the range of e.g. 5 weeks are found to lead to an increase in the regenerative capacities and longer terms of e.g. 10 weeks do not lead to a reduction in the regenerative capacities either. However, best results are achieved with incubation for about 6-8 weeks.

In a particularly preferred embodiment of the invention, SNP is present in the incubation medium in step (a) but not in the cultivation medium in step (c). This further enhances the regeneration-promoting effect of the invention compared to use of SNP in both incubation step (a) and cultivation step (c). Most preferably, SNP is present only in the incubation medium in step (a) but absent during all other steps.

In step (b) of the method of the present invention, plant cells are modified so that they are able to express a polypeptide derived from Brassica napus BBM comprising the amino acid sequence of SEQ ID NO:2 or an amino acid sequence at least 80% identical thereto. A sequence with at least 80% homology may also be a truncated sequence, with truncation preferably occurring at the N-terminus. It was found that truncation at the N-terminus somewhat reduces but does not completely prevent the functionality of BBM for the purposes of the invention and at the same time it brings advantages due to the shorter sequence. The shortening at the N-terminus can, for example, be 1-161 amino acids compared to SEQ ID NO: 2. For example, the N-terminal amino acids 1-161, 1-160, 1-155, or any shorter sub-sections can be deleted.

For modification with BBM, an expression cassette containing a respective coding nucleic acid sequence is inserted directly into the cells to be used in step (a) or into the at least one cell of the callus obtained in step (a). Particularly good results are achieved if first callus tissue is induced in step (a) with SNP as a media component and subsequently in step (b) the obtained callus is transformed with BnBBM. With this two step process, it is possible to obtain shoots even from recalcitrant plant genotypes, for example from Beta vulgaris, Zea mays or Spinacia oleracea.

The coding nucleotide sequence to be used in step (b) comprises
(i) a nucleotide sequence of SEQ ID NO: 1, or a sequence which is at least 80%, preferably at least 85%, at least 90%, more preferably at least 95%, at least 98% or at least 99% identical to the sequence of SEQ ID NO: 1; or
(ii) a nucleotide sequence encoding a polypeptide having the amino acid sequence of SEQ ID NO: 2, or an amino acid sequence which is at least 80%, preferably at least 85%, at least 90%, more preferably at least 95%, at least 98% or at least 99% identical to the sequence of SEQ ID NO: 2.

The above polynucleotides and polypeptides are referred to as “BnBBM-derived nucleic acids” or “BnBBM-derived polypeptides” for simplicity. However, this does not mean that the compounds are obtained from BnBBM, but only that their sequences are derived from the sequence of BnBBM. The way in which the polynucleotides and polypeptides are obtained is basically not restricted.

An aspect of the invention relates to a method of promoting somatic embryogenesis or organogenesis of a plant, comprising the following steps:
(a) inducing callus formation from at least one plant cell,
(b) introducing into the at least one plant cell to be used in step (a) or into the at least one cell of the callus obtained in step (a) an expression cassette comprising a coding nucleotide sequence selected from
(i) a nucleotide sequence which is compared to SEQ ID NO: 1 truncated at the 5′- and/or 3′-end and is at least 80% identical to the sequence of SEQ ID NO: 1, and
(ii) a nucleotide sequence encoding a polypeptide having an amino acid sequence which is truncated compared to SEQ ID NO: 2, preferably N-terminally truncated, and is at least 80% identical to the sequence of SEQ ID NO: 2, wherein the nucleotide sequence is operably linked to a heterologous constitutive regulatory element or a heterologous inducible regulatory element; and
(c) cultivating the callus obtained in step (a) under conditions promoting growth of embryos and/or shoots out of the callus, wherein in the callus the polypeptide is expressed from the expression cassette constitutively or upon induction of the heterologous inducible expression system. According to a preferred embodiment step (a) comprises incubating the at least one plant cell in the presence of sodium nitroprusside (SNP), in particular in a medium comprising SNP. Of course, this aspect of the invention may be further characterized by all features disclosed herein with regard to other aspects of the invention.

For the purpose of this invention, the “sequence identity” of two related nucleotide or amino acid sequences, expressed as a percentage, refers to the number of positions in the two optimally aligned sequences, which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as program NEEDLE as implemented in The European Molecular Biology Open Software Suite (EMBOSS), e.g. version 6.3.1.2 (Trends in Genetics 16 (6), 276 (2000)), with its default parameter, e.g. for proteins matrix=EBLOSUM62, gapopen=10.0 and gapextend=0.5.

The coding nucleotide sequence is operably linked to a heterologous constitutive regulatory element or a heterologous inducible regulatory element. The expression “operably linked” means that said elements of the chimeric gene are linked to one another in such a way that their function is coordinated and allows expression of the coding sequence, i.e. they are functionally linked. By way of example, a promoter is functionally linked to another nucleotide sequence when it is capable of ensuring transcription and ultimately expression of said other nucleotide sequence. Two proteins encoding nucleotide sequences are functionally or operably linked to each other if they are connected in such a way that a fusion protein of first and second protein or polypeptide can be formed.

The heterologous constitutive regulatory element is preferably a constitutive promoter. The heterologous inducible regulatory element can be an inducible promoter or an inducible expression system.

The term “inducible promoter” refers to promoters that selectively express a coding sequence or functional RNA in response to the presence of an endogenous or exogenous stimulus, for example by chemical compounds (chemical inducers) or in response to environmental, hormonal, chemical, and/or developmental signals. Inducible promoters include, for example, promoters induced by light, heat, stress, salt stress, osmotic stress, phytohormones, or chemicals such as ethanol, abscisic acid (ABA), jasmonate, salicylic acid, or safeners.

Promoters useful for targeted expression in trangenesis are reviewed in Potenza at al., 2004 Some abiotic stress promoters are the Arabidopsis thaliana or Oryza sativa DREB genes promoters (Dubouzet et al., 2003; Lee et al., 2004; Pellegrineschi at al., 2004); the Oryza sativa SISAP1, CDPK7 or WSI gene promoters (Mukhopadhyay et al., 2004; Saijo et al., 2000; Takahashi at al., 1994) the A. thaliana rd29 gene promoters (Yamaguchi-Shinozaki and Shinozaki 1993). Some plant heat inducible promoters may also be used hsp18.2 or hsp101 from A. thaliana (Yoshida at al., 1995; Young at al., 2005), hspl7.6 or hspl7.3 from Glycine max (Severin and Schoffl, 1990; Saidi at al., 2005). DNA microarrays have been used to identify stress regulated sequences (Rabbani at al., 2003; EP 1 452 596; WO 02/16655) The signalisation pathway of the response to stress includes abscisic acid signalisation so ABA-inducible promoters may also be powerful stress-inducible promoters, such as the Horgum vulgare A22 and hval promoters (Shen at al., 1993; Straub et al., 1994), Zea maize rab 17, DBF1 and DBF2 (Villardel et al., 1990; Kizis and Pages, 2002), Arabidopsis thaliana ABF3 (Genbank accession AK175851), and Oryza sativa rab21 (Mundy and Chua, 1988).

Some examples of couples of chemically inducible expression systems and chemical inducer used in plants are, the alcA promoter from A. nidulans, inducible by the Ethanol (Rosian at al., 2001) or the ecdysone receptor from C. fumiferana, inducible by an ecdysone agonist (Koo at al., 2004).

In another embodiment, expression of the polypeptide from the coding nucleotide sequence is indirectly induced by a chemical. As an illustration, one can use the GVG gene, which codes for a modified rat glucocorticoid responsive transcription factor that remains in the plant cytosol as a complex. On dexamethasone application, this complex dissociates such that the GVG protein enters the nucleus and binds to the target DNA sequences (UAS). Transcription from the UAS promoter allows the production of the polypeptide. This is considered as a dexamethasone inducible (although indirectly) promoter used to control the polypeptide expression (Aoyama and Chua (1997)). In this case, application of dexamethosone will induce both the expression of the polypeptide and its activity. Preferably, the inducible expression system is selected from an ecdysone or a dexamethasone based expression system.

A gene is said to be expressed when it leads to the formation of an expression product. An expression product denotes an intermediate or end product arising from the transcription and optionally translation of the nucleic acid, DNA or RNA, coding for such product, e. g. the second nucleic acid described herein. During the transcription process, a DNA sequence under control of regulatory regions, particularly the promoter, is transcribed into an RNA molecule. An RNA molecule may either itself form an expression product or be an intermediate product when it is capable of being translated into a peptide or protein. A gene is said to encode an RNA molecule as expression product when the RNA as the end product of the expression of the gene is, e.g., capable of interacting with another nucleic acid or protein. Examples of RNA expression products include inhibitory RNA such as e.g. sense RNA (co-suppression), antisense RNA, ribozymes, miRNA or siRNA, mRNA, rRNA and tRNA. A gene is said to encode a protein as expression product when the end product of the expression of the gene is a protein or peptide.

A nucleic acid (molecule) or nucleotide (sequence) or polynucleotide, as used herein, refers to both DNA and RNA. DNA also includes cDNA and genomic DNA. A nucleic acid molecule can be single- or double-stranded, and can be synthesized chemically or produced by biological expression in vitro or even in vivo.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

As used herein “comprising” or the like is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

In step (b), an expression cassette comprising a coding nucleotide sequence is introduced into the at least one plant cell to be used in step (a) or into the at least one cell of the callus obtained in step (a). Step (b) of introducing into a plant cell the expression cassette comprising a coding nucleotide sequence may result in a stable integration thereof into the genome of the plant cell or a progeny cell thereof. Alternatively, introducing into a plant cell the expression cassette may result in a transient occurrence of the encoded polypeptide in the plant cell or in a progeny cell thereof. In terms of the invention, “transient transformation” means that the inserted sequence is not (stably) integrated into the genome of the plant cell. According to the invention, a stable integration into the genome is preferred.

Introducing the expression cassette can be carried out by any means. A number of methods is available to transfer nucleic acids of interest into plant cells. An exemplary vector mediated method is Agrobacterium-mediated transformation, as described, for example, by Lindsay & Gallois, 1990, Journal of Experimental Botany, and Kischenko et al., 2005, Cell Biology International for sugar beet, or by Ishida et al., 2007, (“Agrobacterium-mediated transformation of maize.” Nature protocols, 2(7), 1614-1621) for corn. Other suitable techniques include particle bombardment and electroporation.

In step (c), cultivation takes place under conditions in which the polypeptide is expressed. Cultivating is preferably carried out in a medium free of plant hormones.

The term “plant hormone” is to be understood herein as a chemical that influences the growth and development of plant cells and tissues. Plant growth hormones comprise chemicals from the following five groups: auxins, cytokinins, gibberellins, abscisic acid (ABA) and ethylene. In addition to the five main groups, two other classes of chemical are often regarded as plant growth regulators: brassinosteroids and polyamines. For the induction of regeneration in plant tissues, a combination of one or more cytokinins and one or more auxins is usually employed. According to the invention, however, plant hormones are preferably not used in the culturing step (c). It was found, that in the absence of plant hormones in the medium, negative side-effects on plant development (e.g. somaclonal variation) are reduced. Hence, the complexity of adjusting medium components to different genotypes is also reduced.

Additionally, it was found that the absence of plant hormones in step (c) promotes the formation of somatic embryos instead of shoots. Thus, in a preferred embodiment, embryos are directly grown from the callus tissue in step (c). Accordingly, a tedious rooting procedure is avoided and hence the time to go to the green house is shortened.

The enhanced somatic embryo formation by using BnBBM in a hormone free transformation—leading to plant development without obvious phenotypes—allows fast validation of trait candidate genes especially for root traits and root tissue specific promoters. It can also be combined with co-delivery of other genetic elements and the application of the genome editing technology. Furthermore, somaclonal variation might be reduced if no hormones like 2,4D are used, especially when the callus tissue is cultured for a longer time period.

A remarkable time saving is the second improvement for plant transformation. In the described approach T0 plants can be directly used for different kind of analyses, whereas normally T1 plants are used in conventional transformation methods for such analyses. This is necessary since the conventional TO plants produced via organogenesis have an obvious phenotype with not forming proper tap roots, the specific storage organ of sugar beet.

Due to the usage of the T0 plants in the described invention up to 2.5 years can be saved for the above-mentioned analyses processes.

Due to the increased regenerative capacities of plant material with inducible as well as constitutive expression of a BnBBM-derived polypeptide as described above, also the efficiency of co-delivery of other genetic elements (e.g. genetic engineering components) is enhanced. More transgenic or modified plants can be obtained and otherwise non-transformable genotypes can be transformed or edited. Thus, according to another aspect of the invention, the combined beneficial effects of SNP and the BnBBM-derived polypeptide as described above can be exploited in methods of producing transgenic plants as well as in methods for producing genetically modified/edited plants. It was found, that in recalcitrant plant species or plant genotypes, transformation efficiency can be improved by carrying out callus induction in the presence of SNP and introducing a BnBBM-derived polypeptide as described above. The increased regenerative capacities of plant tissue with inducible as well as constitutive expression of the BnBBM-derived polypeptide leads also to a more efficient co-transformation rate which is for example important for the delivery of genome editing components. This has been successfully demonstrated by obtaining SDN 1 events. The regeneration of plants from modified plant cells that have been transformed or gene edited and possibly have a modified genome is significantly improved when a BnBBM-derived polypeptide as defined herein is present in the step of cultivating callus tissue. Further, the approach of the invention enables a remarkable time saving which is particularly pronounced when creating edited Beta vulgaris hybrids. Up to 6-7 years may be saved, since the editing event can be produced directly within the two parental lines of the final hybrid, instead of introducing the necessary modification in an amenable genotype for transformation first and afterwards bringing it to the parental lines of a specific hybrid via time consuming crossing and breeding steps.

According to this embodiment, the method further comprises a step of
(d1) introducing at least one nucleotide sequence of interest into the at least one plant cell or a predecessor thereof to be used in step (b), or into at least one cell of the callus obtained in step (a) which itself or a progeny thereof is then to be used in step (b) or has been used in step (b), and/or
(d2) modifying the genome of the at least one plant cell or a predecessor thereof to be used in step (a), or of the at least one cell of the callus obtained in step (a) which itself or a progeny thereof is then to be used in step (b) or has been used in step (b),
   by introducing into said cell a single stranded DNA break (SSB) inducing enzyme or a double stranded DNA break (DSB) inducing enzyme which preferably recognizes a predetermined site in the genome of said cell, and optionally a repair nucleic acid molecule, or a single stranded DNA break (SSB) inducing enzyme which preferably recognizes a predetermined site in the genome of said cell and is fused to a base editor enzyme,
   wherein the modification of said genome is selected from
   I. a replacement of at least one nucleotide;
   II. a deletion of at least one nucleotide;
   III. an insertion of at least one nucleotide; or
   IV. any combination of I.-III.

Step (d1) of introducing the at least one nucleotide sequence of interest can be performed using any suitable method commonly known in the art. A number of methods is available to transfer nucleic acids of interest into plant cells. An exemplary vector mediated method is Agrobacterium-mediated transformation, as described, for example, by Lindsay & Gallois, 1990, Journal of Experimental Botany, and Kischenko et al., 2005, Cell Biology International for sugar beet, or by Ishida et al., 2007, (“Agrobacterium-mediated transformation of maize.” Nature protocols, 2(7), 1614-1621) for corn. Other suitable techniques include particle bombardment and electroporation.

The nucleotide sequence of interest according to the invention may be a DNA or RNA sequence, e.g. mRNA, siRNA, miRNA etc. More particularly, the nucleotide sequence of interest encodes at least one phenotypic trait. Preferably, the phenotypic trait conferred by the DNA or RNA can be selected from the group consisting of resistance/tolerance to biotic stress, including pathogen resistance/tolerance, wherein the pathogen can be a virus, bacterial, fungal or animal pathogen, resistance/tolerance to abiotic stress including chilling resistance/tolerance, drought stress resistance/tolerance, osmotic resistance/tolerance, heat stress resistance/tolerance, cold or frost stress resistance/tolerance, oxidative stress resistance/tolerance, heavy metal stress resistance/tolerance, salt stress or water logging resistance/tolerance, lodging resistance/tolerance, shattering resistance/tolerance, or resistance/tolerance against one or more herbicides like glyphosate, glufosinate, 2,4-D, Dicamba, ALS inhibitors et cetera. The at least one phenotypic trait of interest can also be selected from the group consisting of the modification of a further agronomic trait of interest including yield increase, flowering time modification, seed color modification, endosperm composition modification, nutritional content modification or metabolic engineering of a pathway of interest.

According to one embodiment of the invention, step (d1) of introducing the at least one nucleotide sequence of interest yields in transient transformation of the plant cell. In another embodiment, a stable transformation is effected, wherein the nucleotide sequence of interest in step (d1) is inserted into the genome of the plant cell.

In step (d2), modifying the genome of the plant cell can be accomplished by means of a single stranded DNA break (SSB) or double stranded DNA break (DSB) inducing enzyme or a base editor enzyme which preferably recognizes a predetermined site in the genome of said cell.

As used herein, a “double-stranded DNA break inducing enzyme” or “DSBI enzyme” is an enzyme capable of inducing a double-stranded DNA break at a particular nucleotide sequence, called the “recognition site”. Accordingly, a “single-stranded DNA or RNA break inducing enzyme” or “SSBI enzyme” is an enzyme capable of inducing a single-stranded DNA or RNA break at a particular nucleotide sequence.

In order to enable a break at a predetermined target site, the enzymes preferably include a binding domain and a cleavage domain. Particular enzymes capable of inducing double or single-stranded breaks are nucleases as well as variants thereof, no longer comprising a nuclease function but rather operating as recognition molecules in combination with another enzyme. In recent years, many suitable nucleases, especially tailored endonucleases have been developed comprising meganucleases, zinc finger nucleases, TALE nucleases, Argonaute nucleases, derived, for example, from Natronobacterium gregoryi, and CRISPR nucleases, comprising, for example, Cas, Cpf1, CasX or CasY nucleases as part of the Clustered Regularly Interspaced Short Palindromic Repeats (CRISPR) system. Thus, in a preferred aspect of the invention, the DSB or SSB inducing enzyme is selected from CRISPR systems like CRISPR/Cas9, CRISPR/Cpf1, CRISPR/CasX, CRISPR/CasY, CRISPR/Csm1 or CRISPR/MAD7, preferably a CRISPR/Cas9 endonuclease, a CRISPR/MAD7 endonuclease or a CRISPR/Cpf1 endonuclease, a zinc finger nuclease (ZFN), a homing endonuclease, a meganuclease and a TAL effector nuclease.

Rare-cleaving endonucleases are enzymes that have a recognition site of preferably about 14 to 70 consecutive nucleotides, and therefore have a very low frequency of cleaving, even in larger genomes such as most plant genomes. Homing endonucleases, also called meganucleases, constitute a family of such rare-cleaving endonucleases. They may be encoded by introns, independent genes or intervening sequences, and present striking structural and functional properties that distinguish them from the more classical restriction enzymes, usually from bacterial restriction-modification Type II systems. Their recognition sites have a general asymmetry which contrast to the characteristic dyad symmetry of most restriction enzyme recognition sites. Several homing endonucleases encoded by introns or inteins have been shown to promote the homing of their respective genetic elements into allelic intronless or inteinless sites. By making a site-specific double strand break in the intronless or inteinless alleles, these nucleases create recombinogenic ends, which engage in a gene conversion process that duplicates the coding sequence and leads to the insertion of an intron or an intervening sequence at the DNA level. A list of other rare cleaving meganucleases and their respective recognition sites is provided in Table I of WO 03/004659 (pages 17 to 20) (incorporated herein by reference).

Furthermore, methods are available to design custom-tailored rare-cleaving endonucleases that recognize basically any target nucleotide sequence of choice. Briefly, chimeric restriction enzymes can be prepared using hybrids between a zinc-finger domain designed to recognize a specific nucleotide sequence and the non-specific DNA-cleavage domain from a natural restriction enzyme, such as Fokl. Such methods have been described e.g. in WO 03/080809, WO 94/18313 or WO 95/09233 and in Isalan et al. (2001). A rapid, generally applicable method to engineer zinc fingers illustrated by targeting the HIV-1 promoter. Nature biotechnology, 19(7), 656; Liu et al. (1997). Design of polydactyl zinc-finger proteins for unique addressing within complex genomes. Proceedings of the National Academy of Sciences, 94(11), 5525-5530.).

Another example of custom-designed endonucleases includes the TALE nucleases (TALENs), which are based on transcription activator-like effectors (TALEs) from the bacterial genus Xanthomonas fused to the catalytic domain of a nuclease (e g. Fok/or a variant thereof). The DNA binding specificity of these TALEs is defined by repeat-variable di-residues (RVDs) of tandem-arranged 34/35-amino acid repeat units, such that one RVD specifically recognizes one nucleotide in the target DNA. The repeat units can be assembled to recognize basically any target sequences and fused to a catalytic domain of a nuclease create sequence specific endonucleases (see e g. Boch et al. (2009). Breaking the code of DNA binding specificity of TAL-type Ill effectors. Science, 326(5959), 1509-1512; Moscou & Bogdanove (2009). A simple cipher governs DNA recognition by TAL effectors. Science, 326(5959), 1501-1501; and WO 2010/079430, WO 2011/072246, WO 2011/154393, WO 2011/146121, WO 2012/001527, WO 2012/093833, WO 2012/104729, WO 2012/138927, WO 2012/138939). WO 2012/138927 further describes monomeric (compact) TALENs and TALEs with various catalytic domains and combinations thereof.

Recently, a new type of customizable endonuclease system has been described; the so-called CRISPR/Cas system. A CRISPR system in its natural environment describes a molecular complex comprising at least one small and individual non-coding RNA in combination with a Cas nuclease or another CRISPR nuclease like a Cpf1 nuclease (Zetsche et al., “Cpf1 Is a Single RNA-Guides Endonuclease of a Class 2 CRISPR-Cas System”, Cell, 163, pp. 1-13, October 2015) which can produce a specific DNA double-stranded break. Presently, CRISPR systems are categorized into 2 classes comprising five types of CRISPR systems, the type II system, for instance, using Cas9 as effector and the type V system using Cpf1 as effector molecule (Makarova et al., Nature Rev. Microbiol., 2015). In artificial CRISPR systems, a synthetic non-coding RNA and a CRISPR nuclease and/or optionally a modified CRISPR nuclease, modified to act as nickase or lacking any nuclease function, can be used in combination with at least one synthetic or artificial guide RNA or gRNA combining the function of a crRNA and/or a tracrRNA (Makarova et al., 2015, supra). The immune response mediated by CRISPR/Cas in natural systems requires CRISPR-RNA (crRNA), wherein the maturation of this guiding RNA, which controls the specific activation of the CRISPR nuclease, varies significantly between the various CRISPR systems, which have been characterized so far. Firstly, the invading DNA, also known as a spacer, is integrated between two adjacent repeat regions at the proximal end of the CRISPR locus. Type II CRISPR systems code for a Cas9 nuclease as key enzyme for the interference step, which system contains both a crRNA, and also a trans-activating RNA (tracrRNA) as the guide motif. These hybridize and form double-stranded (ds) RNA regions, which are recognized by RNAseIII and can be cleaved in order to form mature crRNAs. These then in turn associate with the Cas molecule in order to direct the nuclease specifically to the target nucleic acid region. Recombinant gRNA molecules can comprise both the variable DNA recognition region and also the Cas interaction region and thus can be specifically designed, independently of the specific target nucleic acid and the desired Cas nuclease. As a further safety mechanism, PAMs (protospacer adjacent motifs) must be present in the target nucleic acid region, these are DNA sequences, which follow on directly from the Cas9/RNA complex-recognized DNA. The PAM sequence for the Cas9 from Streptococcus pyogenes has been described to be “NGG” or “NAG” (Standard IUPAC nucleotide code) (Jinek et al, “A programmable dual-RNA-guided DNA endonuclease in adaptive bacterial immunity”, Science 2012, 337:816-821). The PAM sequence for Cas9 from Staphylococcus aureus is “NNGRRT” or “NNGRR(N)”. Further variant CRISPR/Cas9 systems are known. Thus, a Neisseria meningitidis Cas9 cleaves at the PAM sequence NNNNGATT. A Streptococcus thermophilus Cas9 cleaves at the PAM sequence NNAGAAW. Recently, a further PAM motif NNNNRYAC has been described for a CRISPR system of Campylobacter (WO 2016/021973 A1). For Cpf1 nucleases it has been described that the Cpf1-crRNA complex, without a tracrRNA, efficiently recognize and cleave target DNA proceeded by a short T-rich PAM in contrast to the commonly G-rich PAMs recognized by Cas9 systems (Zetsche et al., supra). Furthermore, by using modified CRISPR polypeptides, specific single-stranded breaks can be obtained. The combined use of Cas nickases with various recombinant gRNAs can also induce highly specific DNA double-stranded breaks by means of double DNA nicking. By using two gRNAs, moreover, the specificity of the DNA binding and thus the DNA cleavage can be optimized. Further CRISPR effectors like CasX and CasY effectors originally described for bacteria, are meanwhile available and represent further effectors, which can be used for genome engineering purposes (Burstein et al., “New CRISPR-Cas systems from uncultivated microbes”, Nature, 2017, 542, 237-241).

The cleavage site of a DSBI/SSBI enzyme relates to the exact location on the DNA or RNA where the double-stranded break is induced. The cleavage site may or may not be comprised in (overlap with) the recognition site of the DSBI/SSBI enzyme and hence it is said that the cleavage site of a DSBI/SSBI enzyme is located at or near its recognition site. The recognition site of a DSBI/SSBI enzyme, also sometimes referred to as binding site, is the nucleotide sequence that is (specifically) recognized by the DSBI/SSBI enzyme and determines its binding specificity. For example, a TALEN or ZNF monomer has a recognition site that is determined by their RVD repeats or ZF repeats respectively, whereas its cleavage site is determined by its nuclease domain (e.g. Fokl) and is usually located outside the recognition site. In case of dimeric TALENs or ZFNs, the cleavage site is located between the two recognition/binding sites of the respective monomers, this intervening DNA or RNA region where cleavage occurs being referred to as the spacer region.

A person skilled in the art would be able to either choose a DSBI/SSBI enzyme recognizing a certain recognition site and inducing a DSB or SSB at a cleavage site at or in the vicinity of the preselected/predetermined site or engineer such a DSBI/SSBI enzyme. Alternatively, a DSBI/SSBI enzyme recognition site may be introduced into the target genome using any conventional transformation method or by crossing with an organism having a DSBI/SSBI enzyme recognition site in its genome, and any desired nucleic acid may afterwards be introduced at or in the vicinity of the cleavage site of that DSBI/SSBI enzyme.

A “base editor enzyme” or “base” editor as used herein refers to a protein or a fragment thereof having the same catalytical activity as the protein it is derived from, which protein or fragment thereof, alone or when provided as molecular complex, referred to as base editing complex herein, has the capacity to mediate a targeted base modification, i.e., the conversion of a base of interest resulting in a point mutation of interest which in turn can result in a targeted mutation, if the base conversion does not cause a silent mutation, but rather a conversion of an amino acid encoded by the codon comprising the position to be converted with the base editor. Preferably, the at least one base editor according to the present invention is temporarily or permanently linked to at least one site-specific effector, or optionally to a component of at least one site-specific effector complex. The linkage can be covalent and/or non-covalent.

Any base editor or site-specific effector, or a catalytically active fragment thereof, or any component of a base editor complex or of a site-specific effector complex as disclosed herein can be introduced into a cell as a nucleic acid fragment, the nucleic acid fragment representing or encoding a DNA, RNA or protein effector, or it can be introduced as DNA, RNA and/or protein, or any combination thereof.

There are two major and distinct pathways to repair breaks—homologous recombination and non-homologous end-joining (NHEJ). Homologous recombination requires the presence of a homologous sequence as a template (e.g., “donor”) to guide the cellular repair process and the results of the repair are error-free and predictable. In the absence of a template (or “donor”) sequence for homologous recombination, the cell typically attempts to repair the break via the process of non-homologous end-joining (NHEJ).

In a particularly preferred aspect of this embodiment, a repair nucleic acid molecule is additionally introduced into the plant cell. As used herein, a “repair nucleic acid molecule” is a single-stranded or double-stranded DNA molecule or RNA molecule that is used as a template for modification of the genomic DNA at the preselected site in the vicinity of or at the cleavage site. As used herein, “use as a template for modification of the genomic DNA”, means that the repair nucleic acid molecule is copied or integrated at the preselected site by homologous recombination between the flanking region(s) and the corresponding homology region(s) in the target genome flanking the preselected site, optionally in combination with non-homologous end-joining (NHEJ) at one of the two end of the repair nucleic acid molecule (e.g. in case there is only one flanking region). Integration by homologous recombination will allow precise joining of the repair nucleic acid molecule to the target genome up to the nucleotide level, while NHEJ may result in small insertions/deletions at the junction between the repair nucleic acid molecule and genomic DNA.

As used herein, “a modification of the genome”, means that the genome has changed by at least one nucleotide This can occur by replacement of at least one nucleotide and/or a deletion of at least one nucleotide and/or an insertion of at least one nucleotide, as long as it results in a total change of at least one nucleotide compared to the nucleotide sequence of the preselected genomic target site before modification, thereby allowing the identification of the modification, e.g. by techniques such as sequencing or PCR analysis and the like, of which the skilled person will be well aware.

As used herein “a preselected site”, “a predetermined site” or “predefined site” indicates a particular nucleotide sequence in the genome (e.g. the nuclear genome or the chloroplast genome) at which location it is desired to insert, replace and/or delete one or more nucleotides. This can e.g. be an endogenous locus or a particular nucleotide sequence in or linked to a previously introduced foreign DNA or transgene. The preselected site can be a particular nucleotide position at (after) which it is intended to make an insertion of one or more nucleotides. The preselected site can also comprise a sequence of one or more nucleotides, which are to be exchanged (replaced) or deleted.

As used in the context of the present application, the term “about” means+/−10% of the recited value, preferably +/−5% of the recited value. For example, about 100 nucleotides (nt) shall be understood as a value between 90 and 110 nt, preferably between 95 and 105.

As used herein, a “flanking region”, is a region of the repair nucleic acid molecule having a nucleotide sequence which is homologous to the nucleotide sequence of the DNA region flanking (i.e. upstream or downstream) of the preselected site. It will be clear that the length and percentage sequence identity of the flanking regions should be chosen such as to enable homologous recombination between said flanking regions and their corresponding DNA region upstream or downstream of the preselected site. The DNA region or regions flanking the preselected site having homology to the flanking DNA region or regions of the repair nucleic acid molecule are also referred to as the homology region or regions in the genomic DNA.

To have sufficient homology for recombination, the flanking DNA regions of the repair nucleic acid molecule may vary in length, and should be at least about 10 nt, about 15 nt, about 20 nt, about 25 nt, about 30 nt, about 40 nt or about 50 nt in length. However, the flanking region may be as long as is practically possible (e.g. up to about 100-150 kb such as complete bacterial artificial chromosomes (BACs). Preferably, the flanking region will be about 50 nt to about 2000 nt, e.g. about 100 nt, 200 nt, 500 nt or 1000 nt. Moreover, the regions flanking the DNA of interest need not be identical to the homology regions (the DNA regions flanking the preselected site) and may have between about 80% to about 100% sequence identity, preferably about 95% to about 100% sequence identity with the DNA regions flanking the preselected site. The longer the flanking region, the less stringent the requirement for homology. Furthermore, to achieve exchange of the target DNA sequence at the preselected site without changing the DNA sequence of the adjacent DNA sequences, the flanking DNA sequences should preferably be identical to the upstream and downstream DNA regions flanking the preselected site.

As used herein, “upstream” indicates a location on a nucleic acid molecule, which is nearer to the 5′ end of said nucleic acid molecule. Likewise, the term “downstream” refers to a location on a nucleic acid molecule which is nearer to the 3′ end of said nucleic acid molecule. For avoidance of doubt, nucleic acid molecules and their sequences are typically represented in their 5′ to 3′ direction (left to right).

In order to target sequence modification at the preselected site, the flanking regions must be chosen so that 3′ end of the upstream flanking region and/or the 5′ end of the downstream flanking region align(s) with the ends of the predefined site. As such, the 3′ end of the upstream flanking region determines the 5′ end of the predefined site, while the 5 end of the downstream flanking region determines the 3′ end of the predefined site.

As used herein, said preselected site being located outside or away from said cleavage (and/or recognition) site, means that the site at which it is intended to make the genomic modification (the preselected site) does not comprise the cleavage site and/or recognition site of the DSBI/SSBI enzyme or the base editor enzyme, i.e. the preselected site does not overlap with the cleavage (and/or recognition) site. Outside/away from in this respect thus means upstream or downstream of the cleavage (and/or recognition) site.

In the present invention it was found that BnBBM enhances somatic embryo formation in plant transformation and leads to plants with high quality, comparable to plants grown from seeds. The high quality of the obtained plants had been verified by the absence of obvious visible phenotypes but also experimentally for root features e.g. weight, sucrose content as well as for leaf parameters like photosynthesis. Such an increased plant quality was observed after Agrobacterium-based transformation as well as biolistic transformation methods in combination with BnBBM induced somatic embryogenesis.

The enhanced somatic embryo formation by using BnBBM in a hormone free transformation—leading to plant development without obvious phenotypes—allows fast validation of trait candidate genes especially for root traits and root tissue specific promoters, furthermore it could also be combined with the application of the genome editing technology.

A remarkable time saving is the second improvement for plant transformation. In the described approach T0 plants can be directly used for different kind of analyses, whereas normally T1 plants are used in conventional transformation methods to do such analyses. This is necessary since the conventional TO plants produced via organogenesis have an obvious phenotype with not forming proper tap roots, the specific storage organ of sugar beet.

Due to the usage of the T0 plants in the described invention up to 2.5 years can be saved for the above-mentioned analyses processes. Moreover, there is also the possibility of transient genome editing in plants when using TO plants. Accordingly, such a plant analysis at the T0 level represents a preferred embodiment of the present invention.

The time savings for creating genetically engineered Beta vulgaris hybrids are even higher. Up to 6-7 years might be saved, since instead of generating edits in many parental lines of monogerm, edits could directly be generated in the parents for producing the hybrids (FIG. 29). Further improvements of this invention are a less somaclonal variation in plants obtained from this method and a significant increase of seed production of transgenic plants. This is often a major problem, in particular for Beta vulgaris plants, and leads to strong limitation regarding usability of different lines.

A further aspect of the invention is drawn to a method for the production of a plant. It was found, that using the above described methods of somatic embryogenesis and organogenesis, whole plants can be produced even for recalcitrant plant species or plant genotypes. Further, it was found that with the present invention, the resulting plants show no pleiotropic phenotype.

Accordingly, the invention provides a method for producing a plant comprising somatic embryogenesis or organogenesis as described above with reference to steps (a) and (b), optionally transformation or genomic modification as described above with reference to steps (d1) and (d2), and regenerating a plant from an embryo and/or a shoot resulting from step (c) of the above disclosed method, in particular a transgenic plant resulting from step (d1) and/or a modified plant resulting from step (d2).

Preferably, the conditions of somatic embryogenesis are chosen so that embryos are formed directly. This can be achieved as described above, for example by carrying out the cultivation in a medium free of plant hormones. From the shoots derived from somatic embryogenesis in hormone free medium, seedlings are grown having natural roots instead of the adventitious roots that are artificially induced from in vitro shoots which allows physiological analysis already at the TO level. Moreover, the quality of obtained plants is clearly better compared to plants generated with published protocols. This is leading to an enormous time and cost saving due to reduced tissue culture work in comparison with the state of the art. Accordingly, such a physiological analysis at the T0 level represents a preferred embodiment of the present invention.

Subject matter of the present invention are also the plants that are obtained or obtainable by the methods described above. Accordingly, one embodiment of the invention is a transgenic plant obtained or obtainable by the above method of transforming a plant cell and producing a plant, as well as progeny plant or parts thereof, wherein the progeny or the part comprises the at least one nucleotide sequence of interest as transgene.

Another embodiment of the invention is a genetically modified plant obtained or obtainable by the above method of modifying the genome of a plant cell and producing a plant (claim 12) as well as progeny plants or parts thereof, wherein the progeny or the part comprises the modification in the genome introduced by the above method of modification.

Further subject matter of the present invention is a plant cell or a seed derived from the above transgenic plant or genetically modified plant. Such a plant cell preferably comprises a polynucleotide encoding a BnBBM-derived polypeptide transiently or stably integrated and a single-stranded DNA break (SSB)- or double-stranded DNA break (DSB)-inducing enzyme or base editor enzyme, which preferably recognizes a predetermined site in the genome of said cell and optionally a repair nucleic acid molecule. The polynucleotide encoding the BnBBM-derived polypeptide is preferably operably linked to a suitable regulatory sequence so that the plant cell is capable of expressing the BnBBM-derived polypeptide. A regulatory sequence means, for example, a “promoter” which refers to a nucleotide sequence, usually upstream (5) to its coding sequence, which controls the expression of the coding sequence by providing the recognition for RNA polymerase and other factors required for proper transcription. “Constitutive promoter” refers to promoters that direct gene expression in nearly all tissues and at all times. Examples of constitutive promoters include CaMV 35S promoter, double CaMV 35S promoter (70S promoter), nopaline synthase (nos) promoter, BdEF1 promoter, or ubiquitin promoters like PcUbi4 or ZmUbi1. “Regulated promoter” refers to promoters that direct gene expression not constitutively but in a temporally and/or spatially regulated manner and include both tissue-specific and inducible promoters. It includes natural and synthetic sequences as well as sequences, which may be a combination of synthetic and natural sequences. Different promoters may direct the expression of a gene in different tissues or cell types, or at different stages of development, or in response to different environmental conditions. New promoters of various types useful in plant cells are constantly being discovered and are well-known to a person skilled in the art. “Tissue-specific promoter” refers to regulated promoters that are not expressed in all plant cells but only in one or more cell types in specific organs (such as leaves or seeds), specific tissues (such as embryo or cotyledon), or specific cell types (such as leaf parenchyma or seed storage cells). These also include promoters that are temporally regulated (such as in early or late embryogenesis), during fruit ripening in developing seeds or fruit, in fully differentiated leaf, or at the onset of senescence. “Inducible promoter” refers to those regulated promoters that can be turned on in one or more cell types by an external stimulus (such as a chemical, light, hormone, stress, or pathogen). Examples for inducible promoter are promoters inducible by ecdysone, dexamethasone, ethanol. Such promoters are well known from the state of the art (e.g., Samalova et al. (2005). pOp6/LhGR: a stringently regulated and highly responsive dexamethasone-inducible gene expression system for tobacco. The Plant Journal, 41(6), 919-935; Gatz & Lenk (1998). Promoters that respond to chemical inducers. Trends in Plant Science, 3(9), 352-358.).

Another subject matter of the present invention is a plant cell comprising a polynucleotide encoding a BnBBM-derived polypeptide transiently or stable integrated, and a single-stranded DNA break (SSB) or double stranded DNA break (DSB) inducing enzyme or base editor enzyme which preferably recognize a predetermined site in the genome of said cell, and optionally a repair nucleic acid molecule, wherein preferably the polynucleotide encoding the BnBBM-derived polypeptide being operatively linked to a suitable regulatory sequence, so that the plant cell is capable of expressing the polypeptide. Such plant cell can be obtained when conducting the above described method for modifying the genome of a plant cell.

Another aspect of the invention is the use of SNP in combination with a nucleic acid encoding a BnBBM-derived polypeptide as described above in a method for somatic embryogenesis or organogenesis and/or plant regeneration from callus. In particular, the invention discloses the use of SNP in combination with a nucleic acid comprising a coding nucleotide sequence selected from
(i) a nucleotide sequence of SEQ ID NO: 1, or a sequence which is at least 80% identical to the sequence of SEQ ID NO: 1; and
(ii) a nucleotide sequence encoding a polypeptide having the amino acid sequence of SEQ ID NO: 2, or an amino acid sequence which is at least 80% identical to the sequence of SEQ ID NO: 2
in a method for somatic embryogenesis or organogenesis and/or plant regeneration of Beta vulgaris, Zea mays or Spinacia oleracea from callus; in a method for direct or indirect regeneration of a Beta vulgaris plant, Zea mays plant or Spinacia oleracea plant;
in a method of transformation of a Beta vulgaris, Zea mays or Spinacia oleracea plant cell; or
in a method of modifying the genome of a Beta vulgaris, Zea mays or Spinacia oleracea plant cell.

According to a further aspect of the invention, the activity of SNP and a BnBBM-derived polypeptide in the above described methods and uses can be combined with a polynucleotide encoding a WUS2 polypeptide, mRNA encoding a WUS2 polypeptide, or WUS2 polypeptide(s). Particularly preferred is Zea mays WUS2 (ZmWUS2; cDNA; SEQ ID NO: 12; protein: SEQ ID NO: 13).

Accordingly, in addition to introducing an expression cassette comprising a polynucleotide encoding a BBM-derived polypeptide, the approach of the invention according to this aspect further provides for introducing an expression cassette comprising a polynucleotide encoding a WUS2 polypeptide, mRNA encoding a WUS2 polypeptide, or WUS2 polypeptide(s) into the at least one plant cell concerned. The polynucleotide encoding a WUS2 polypeptide can be integrated into the same expression cassette and operably linked to the same regulatory elements as the BnBBM-derived polynucleotide.

WUS2 can be provided to the at least one plant cell concerned as an expression cassette comprising a second coding nucleotide sequence
(i) having a nucleotide sequence of SEQ ID NO: 12, or a sequence which is at least 80% identical to the sequence of SEQ ID NO: 12; or
(ii) encoding a polypeptide having the amino acid sequence of SEQ ID NO: 13, or a sequence which is at least 80% identical to the sequence of SEQ ID NO: 13,
wherein the second nucleotide sequence is operably linked to a heterologous regulatory element.

Further, WUS2 can be provided to the at least one plant cell as an mRNA encoding a polypeptide having the amino acid sequence of SEQ ID NO: 13, or a sequence, which is at least 80% identical to the sequence of SEQ ID NO: 13.

WUS2 can also be introduced into the at least one plant cell as a polypeptide having the amino acid sequence of SEQ ID NO: 13, or a sequence, which is at least 80% identical to the sequence of SEQ ID NO: 13.

Further, an enhanced expression level of an endogenous gene encoding the polypeptide having the amino acid sequence of SEQ ID NO: 13, or a sequence which is at least 80% identical to the sequence of SEQ ID NO: 13 can be induced in the at least one plant cell. This leads to an enhanced expression level of the endogenous WUS2 gene, i.e. to the presence or occurrence of WUS2 polypeptide in an enhanced amount in the plant cell.

The activation of the expression of the endogenous gene can be achieved by modifying the activity or structure of the promoter of the endogenous gene encoding the WUS2 polypeptide. For instances, enhancer elements can be introduced into the promoter by means of gene editing; or either an enhancer element regulating the promoter can be further strengthen or a silencer element regulating the promoter can be weakened by e.g. targeted mutagenesis/modification; or modifications can be introduced into the epigenome related to enhancers by means of gene editing tools like CRISPR systems (Hilton et al. (2015). Epigenome editing by a CRISPR-Cas9-based acetyltransferase activates genes from promoters and enhancers. Nature biotechnology, 33(5), 510-517); or synthetic transcription factors based on e.g. TALE activators or dCas9 activators can be introduced into the cell where they are able to bind targeted recognition sites on or near by the promoter und activate transcription of the WUS2 gene (Cheng et al. (2013) Multiplexed activation of endogenous genes by CRISPR-on, an RNA-guided transcriptional activator system. Cell research, 23(10), 1163.); or the amount of microRNA (miRNA) in the plant cell regulating the expression of the WUS2 gene by post-transcriptional inhibition can be reduced by e.g. knock out (null mutant) or knock down in order to increase the amount of translated WUS2 polypeptide in the plant cell.

Dependent on the plant species as well as on the cell type different levels of gene or expression activation are needed in order to have adequate amount of WUS2 polypeptide present in the plant cell at the time when regeneration takes place.

There are various techniques available to a person skilled in the art in order to measure the actual expression level of an endogenous or an introduced gene, e.g., qPCR, RT-PCR, Northern blot, or microrarrays. These methods allow those skilled in the art by routine work to adjust the level of expression of the WUS2 gene which effects improved regeneration ability from diverse tissues or somatic and reproductive cells (e.g. microspores). In a preferred embodiment, in the plant cell the expression level of an endogenous gene encoding a WUS2 polypeptide is increased at least by the factor of 2, the factor of 3, or the factor of 5, preferably by the factor of 10, the factor of 25 or factor of 50, more preferred by the factor of 100, the factor of 200, or the factor of 500.

As described further above the induction of an enhanced expression level of an endogenous gene in a plant cell can be carried out by the application of one or more activators or a precursor thereof. These can be applied to the medium in which the plant cells are cultivated and is then actively or passively absorbed by the plant cell. Furthermore, the one or more activator or a precursor thereof can be directly introduced into the plant cell by microinjection, electroporation or biolistic bombardment. Beside the above synthetic transcription activators, a number of further activators are known from the state of the art that can be used for increasing the expression level of an endogenous gene, in particular the expression level of the endogenous WUS2 gene: In the recent years, the technical fields of chemical plant genetics and chemical plant biology emerged where biological systems are treated with small molecules to specifically perturb cellular functions. Small molecules are used commercially as drugs, herbicides, and fungicides in different systems, but in recent years they are increasingly exploited also as tools for genetic regulation. For instance, chemical genetics involves the discovery of small-molecule effectors of various cellular functions through screens of compound libraries (Dejonghe & Russinova (2017). Plant Chemical Genetics: From Phenotype-Based Screens to Synthetic Biology. Plant Physiology, pp-01805; Kawasumi, M., & Nghiem, P. (2007). Chemical genetics: elucidating biological systems with small-molecule compounds. Journal of Investigative Dermatology, 127(7), 1577-1584.). Such small molecule effectors suitable for the activation of the expression of a target gene like WUS2, can be identified by chemical screens following different strategies (Dejonghe & Russinova, 2017). Comprehensive compound libraries are available which allow the simple screening of countless small molecules and the identification of effectors which can be used for activation of the gene expression of genes like WUS2. As mentioned above another approach to enhance the expression level of an endogenous gene like WUS2 is the application of so-called synthetical transcription activators. They are typically designed by the fusion of a recognition domain and at least one activator domain. The recognition domain can be derived from known systems like Zinc finger, TAL effectors or CRISPR; for activation, fusing for instances the herpes simplex virus derived VP-16 or VP-64 activation domains to a recognition domain can cause an increase in transcription. Weaker activation domains such as the AD of human NF-κB add to the variety of options for gene activation. Furthermore, as shown on endogenous promoters, combinations of activators can be used to introduce synergistic effects (Moore et al. (2014). “Transcription activator-like effectors: a toolkit for synthetic biology.” ACS synthetic biology, 3(10), 708-716.; US 2002/0046419 A1; Lowder et al. (2017). “Multiplexed transcriptional activation or repression in plants using CRISPR-dCas9-based systems.” Plant Gene Regulatory Networks: Methods and Protocols, 167-184.). The synthetical transcription activator can be delivered to the plant cell or introduced into the plant cell also as precursor, i.e. as DNA or RNA molecule encoding such artificial or synthetical transcription activator or a domain thereof or as inactive form of transcription activator which is activated later in the cell or a in a specific compartment of the cell. Finally, enhancing expression of WUS2 genes can be also achieved by the inactivation of upstream negative regulators or by the creation of a mutant version of the BBM gene that is resistant to such negative regulators. According to the above described aspects, the invention further provides for the following step (e) to be carried out on at least one plant cell to be used in step (a) or at least one cell of the callus obtained in step (a) of the above described methods:
(e) introducing into the at least one plant cell
   (I) an expression cassette comprising a second nucleotide sequence
      (i) having a polypeptide coding sequence of SEQ ID NO: 12, or a sequence which is at least 80% identical to the sequence of SEQ ID NO: 12, preferably at least 85%, at least 90%, more preferably at least 95%, at least 98% or at least 99% identity to SEQ ID NO: 12; or
      (ii) encoding a polypeptide having the amino acid sequence of SEQ ID NO: 13, or a sequence which is at least 80% identical to the sequence of SEQ ID NO: 13, preferably at least 85%, at least 90%, more preferably at least 95%, at least 98% or at least 99% identity to SEQ ID NO: 13,
      wherein the second nucleotide sequence is operably linked to the first heterologous regulatory element or to a second heterologous regulatory element; or
   (II) an mRNA encoding a polypeptide having the amino acid sequence of SEQ ID NO: 13, or a sequence, which is at least 80% identical to the sequence of SEQ ID NO: 13, preferably at least 85%, at least 90%, more preferably at least 95%, at least 98% or at least 99% identity to SEQ ID NO: 13; or
   (III) a polypeptide having the amino acid sequence of SEQ ID NO: 13, or a sequence, which is at least 80% identical to the sequence of SEQ ID NO: 13, preferably at least 85%, at least 90%, more preferably at least 95%, at least 98% or at least 99% identity to SEQ ID NO: 13; or
   (IV) inducing in said at least one plant cell an enhanced expression level of an endogenous gene encoding the polypeptide having the amino acid sequence of SEQ ID NO: 13, or a sequence which is at least 80% identical to the sequence of SEQ ID NO: 13, preferably at least 85%, at least 90%, more preferably at least 95%, at least 98% or at least 99% identity to SEQ ID NO: 13, and
wherein cultivating the callus in step (c) to promote growth of embryos and/or shoots out of the callus is carried out under conditions wherein in the callus the BnBBM-derived polypeptide is expressed as described above, and the WUS2 polypeptide is expressed from the expression cassette, is translated from introduced mRNA, is enhanced expressed from the endogenous gene, or is present.

The first and the second heterologous regulatory elements can be selected from the group consisting of a constitutive promoter, an inducible promoter or an inducible expression system Introducing of the expression cassette(s) can result in a stable integration thereof into the genome of the at least one plant cell or in a progeny cell thereof.

Another aspect of the invention is a transgenic plant obtained or obtainable by the above method or a progeny plant thereof. Further, the invention provides a plant cell or a seed of said transgenic plant, wherein the plant cell or the seed comprises the at least one nucleotide of interest as transgene.

A still further aspect of the invention is a modified plant obtained or obtainable by the above method, or a progeny plant thereof. Further, the invention provides a plant cell or a seed of said transgenic plant, wherein the plant cell or the seed comprises the modification in the genome.

Cells, plants, progeny plants and plant parts obtained by purely biological processes are not the subject of the invention.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Cray, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

All patents, patent applications, and publications or public disclosures (including publications on internet) referred to or cited herein are incorporated by reference in their entirety.

The invention will be further described with reference to the following Figures and Examples described herein. However, it is to be understood that the invention is not limited to such Examples.

### FIGURES

FIG. 1 Shoot formation in sugar beet with and without DEX inducible BBM-GR expression. A: no shoot formation without application of the DEX inducer; B: Many shoots (arrows) are formed upon treatment with the DEX inducer.

FIG. 2 Dex inducible 35s-BnBBM-GR plants look comparable to control plants regenerated from tissue culture without transgene (control).

FIG. 3 BnBBM increases callus-based transformation efficiency in sugar beet (quantification of transformation rate) in amenable and recalcitrant genotypes. A: inducible system—comparison of percentage regenerated plants between control, BnBBM-GR without DEX Inducer, and BnBBM-GR with DEX Inducer. B: constitutive overexpression—comparison between control and 35S-BnBBM overexpression. Sugar beet plants transformed with Tdt expression construct under a constitutive promoter serves as control in A and B.

FIG. 4 35S-BnBBM (constitutive expression) give rise to embryo (B, arrow shows embryo (e)) and seedlings (C, arrow shows root (r) and seedling (s)) while standard callus based trafo for sugar beet only generates shoots (A; arrow shows shoot derived from organogenesis but no root (s*)). In total may transgenic events can be produced by means of 35S-BnBBM expression (D).

FIG. 5 35S-BnBBM produces mainly embryos on hormone free medium (B) while produces shoots and embryos on normal regeneration medium with hormones (A)

FIG. 6 BnBBM breaks recalcitrance in sugar beet. 35S:BnBBM overexpressing callus of recalcitrant genotype (B) compared with control callus of the same genotype without overexpression of BnBBM (A). Arrows indicate shoots. C: Regenerated transgenic plants of recalcitrant genotype as shown in B.

FIG. 7 Comparison of control construct with vector 70s-tDT versus co-transformation with vector 35S:BnBBM-GR and vector 70s-tDT with inducible BnBBM in two different genotypes (amenable (left) and recalcitrant (right)).

FIG. 8 Constitutive ZmWUS2 overexpression combined with constitutive BnBBM overexpression promotes maize regeneration after biolistic delivery. Arrows indicate fluorescent embryonic structures.

FIG. 9 Vector map of IPR232-pS-01

FIG. 10 Vector map of IPR252-pS-01

FIG. 11 Vector map of IPR 252-pK-02

FIG. 12 In vitro T0 plants regenerated on hormone-free media and with BnBBM show normal root development.

FIG. 13 SNP enhances callus induction in sugar beet amenable genotype 9BS0448. Left column: Callus induction of sugar beet amenable genotype 9BS0448 without SNP (A), with 10 pM SNP (C) and with 100 pM SNP (E); arrows indicate callus induction.
Right column: Close-up view of callus induced without SNP (B), with 10 pM SNP (D) and with 100 pM SNP (F); arrows indicate callus induction.

FIG. 14 SNP enhances callus induction in sugar beet amenable genotype 1 RV6183. Callus induction of sugar beet amenable genotype 1 RV6183 without SNP (A), with 10 pM SNP (B) and with 100 pM SNP (C); arrows indicate callus induction.

FIG. 15 SNP promotes callus induction in sugar beet recalcitrant genotype 8RV6921. Callus induction of sugar beet recalcitrant genotype 8RV6921 without SNP (A), with 10 pM SNP (B) and with 100 pM SNP (C); arrows indicate callus induction.

FIG. 16 Quantification of callus induction of different genotypes. Callus induction rate [%] of sugar beet genotypes 9BS0448, 1 RV6183 and 8RV6921 treated with no SNP (=standard), with 10 mM SNP and with 100 mM SNP.

FIG. 17 Quantification of sugar beet callus shoot induction of different genotypes. Quantification of obtained shoots from callus of sugar beet genotypes 9BS0448 and 1 RV6183 treated with no SNP (=standard), with 10 mM SNP and with 100 mM SNP.

FIG. 18 SNP enhances callus induction in sugar beet genotype 8RR3222. Callus induction of sugar beet genotype 8RR3222 without SNP (A) and with 100 pM SNP (B).

FIG. 19 SNP enhances callus induction in sugar beet genotype 6RR1798 Callus induction of sugar beet genotype 6RR1798 without SNP (A) and with 100 pM SNP (B).

FIG. 20 SNP enhances callus induction in sugar beet genotype 7RV5706 (haploid). Callus induction of sugar beet genotype 7RV5706 (haploid) without SNP (A) and with 100 pM SNP (B)

FIG. 21 Agrobacterium-mediated sugar beet callus transformation.
Pictures taken under a UV light microscope of sugar beet callus which has been treated with 10 pM (A and B) or 100 pM (C and D) and subsequently transformed with pZFN-nptII-70s::tDT by an agrobacterium mediated approach.

FIG. 22 Biolistic delivery of plasmid into the sugar beet callus.
Pictures taken under a UV light microscope of sugar beet callus which has been treated with SNP and subsequently transformed with pZFN-nptII-70s::tDT by a biolistic bombardment approach.

FIG. 23 SNP increases embryogenic callus formation in corn genotype 9D1437. Close-up view of induced callus of corn genotype 9D1437 without SNP (A-C), with 10 pM SNP (D-F) and with 100 pM SNP (G-1).

FIG. 24 Vector map of pZFN-nptII-70s::tDT

FIG. 25 Callus induced with SNP and transformed with BnBBM.
A: Callus of recalcitrant genotype 1T4000 (white arrow) was induced with 100 pM SNP and transformed with BnBBM-GR, subsequently shoot development (black arrow) was observed, indicated by arrows.
B: Quantification of transformation rate [%] of recalcitrant sugar beet genotype 1T4000. Respective callus was induced with or without SNP and subsequently transformed with BnBBM-GR under the control of the 70s promoter. When callus was treated without SNP no transformants were obtained. Whereas when callus was induced with SNP and transformed with BnBBM-GR, shoot development was observed. With this two-step process the obtained transformation rate was 1.67%.

FIG. 26 Influence of SNP on seed production of genotype 9BS0448.
A: Total number of seeds and number of viable seeds are shown for single plants of the sugar beet genotype 9BS0448. These plants were either regenerated from callus which had been induced with 10 pM or 100 pM or obtained conventionally (donor plants).
B: The averages for viable seeds per plants including standard deviations are shown for the different SNP treatments as well as for the control using the donor plants.
Overall the results show a noteworthy reduction of viable seeds in plants obtained from callus which had been induced with a low SNP concentration (10 μM), whereas the callus induction with the higher SNP concentration (100 μM) led to the development of plants producing viable seeds in a quantitative comparable manner to plants which had been obtained conventionally.

FIG. 27 Influence of SNP on seed production of genotype 1 RV6921.
A: Total number of seeds and number of viable seeds are shown for single plants of the sugar beet genotype 1 RV6921. These plants were either regenerated from callus which had been induced with 10 μM or 100 μM or obtained from callus without SNP treatment.
B: The averages for viable seeds per plants including standard deviations are shown for the different SNP treatments as well as for the control plants were no SNP was used for callus induction.
Overall, the results show a reduction of viable seeds in plants obtained from callus, which had been induced with a low SNP concentration (10 μM) in comparison to plants without SNP treatment.
Plants obtained from callus with a high SNP treatment (100 μM) developed remarkable more viable seeds than control plants. This is supporting the data shown in FIG. 26 and verifying the positive effect of SNP treatment on plant development when applied in a sufficient concentration during callus induction.

FIG. 28 Embryogenesis via BnBBM in hormone free media. A: Media with hormones;
B: Media without hormones. By changing media components, i.e. completely removing GA3 and BAP, somatic embryo formation is significantly increased.

FIG. 29 Comparison of seedlings from hormone free media and shoots from media with hormones. A: Regenerated shoot on media with the plant hormones GA3 and BAP; B: regenerated seedling on hormone free media.

FIG. 30 Seedlings (A and B) grown on media without hormones, developing a normal root system, left: top view; right: bottom view FIG. 31 Transgenic 35S-BnBBM-GR plants obtained by particle bombardment.
A: Seedling cultivated in vitro (left: top view; right: bottom view); B: Plantlets after transfer to greenhouse.

FIG. 32 Plant derived from the hormone free protocol develops robust root system.

FIG. 33 Plants derived from the hormone free protocol develops normal upper ground part (one months after transfer to green house)

FIG. 34 Analysis of morphology of tap root (plant 1) after 4 months in the greenhouse

FIG. 36 Analysis of morphology of tap root (plant 2) after 4 months in the greenhouse

FIG. 36 Analysis of transgenic plants from hormone free transformation protocol.
a: Photosynthesis rate in [μE]
b: Sucrose concentration in tap root [μmol/g]

FIG. 37 35S:BnBBM-GR plants are fertile (one-seedling protocol)
a: Seed production in kg per plant
b Viable seed produced per plant

FIG. 38 Comparison of seed production

FIG. 39 Comparison of tap roots after 4 months development in the greenhouse
A: Tap roots from seed derived plants
B: Tap roots from plants obtained by the hormone free transformation

FIG. 40 Tap root parameters of WT seed derived plants and transgenic plants obtained from the hormone free transformation protocol
a: Comparison of tap root weight [g] from seed derived WT and transgenic 35S-BnBBM-GR plants obtained by the hormone free transformation protocol
b: Comparison of sucrose content [μmol/g FW]from seed derived WT and transgenic 35S-BnBBM-GR plants obtained by the hormone free transformation protocol

FIG. 41 Conceptual time saving for field test of SDN-1 hybrids

FIG. 42 Schematic representation of the applied BBM versions and the respective observed regenerative impact The C-terminal BBM deletion does not promote spontaneous regeneration anymore, whereas the N-terminal BBM deletion still induces plant regeneration. Compared to the WT the N-terminal BBM reached a level of approx. 40% of regenerated embryogenic T1 seedlings.

FIG. 43 Phenotypes observed in approx. 10 day-old 35S::BnBBM and BnBBMΔN Arabidopsis seedlings. In both, seedlings can look wild-type (A), show embryogenic tissue at the border of hypocotyl and root (B), develop somatic embryos on cotyledon margins and/or root tip (C), or develop as amorphous clumps of embryogenic tissue (D).
Arrowheads indicate embryogenic tissue.

FIG. 44 Transformation comparison (selection phase: end of C3)
In tissue transformed with an overexpression construct (d35s-BnBBMΔN) shoot and embryo formation was observed [C & D]. This was not the case for the control lines-d35s-tDT [A & B]

FIG. 45 BnBBM N-terminal deletion (d35s-BnBBM-Nter) increases transformation efficiency and triggers embryogenesis
Overexpression of N-terminal deletion version (BnBBMΔN) triggers embryogenesis (A and B).
The regeneration can be both embryogenesis (A and B) and organogenesis (C) The overexpression of BnBBMΔN leads to a clearly increase in transformation efficiency compared to the control line (d35s-tDT) (D)

FIG. 46 Bar diagram illustrating seed production for each individual plant from d35s-BnBBM-Nter
Sugar beet plants transformed with d35s-BnBBMΔN are fertile and produce viable seeds.

### EXAMPLES

### 1. Expression of BnBBM

The principle of the inducible expression of BnBBM is based on a fusion of the rat glucocorticoid receptor domain to the transcription factor BnBBM (BnBBM-GR). Upon induction with Dexamethasone, the GR receptor domain will change conformation, so that the BnBBM-GR protein will enter the nucleus and generate the desired response.

The binary plasmids IPR232-pS-01 and IPR252-pS-01 were produced by KWS with standard cloning procedures. The T-DNA also contains the neomycin phosphotransferase II (nptII) gene that confers resistance to a range of aminoglycoside antibiotics such as kanamycin or paromomycin and was used for the selection of transgenic plant cells and tissues. The NOS promoter and the pAG7 terminator flank the nptII gene. The backbone of the binary vector contains the colE1 and the pVS1 origins for plasmid replication in Escherichia coli and Agrobacterium tumefaciens, respectively; and the aadA gene that confers streptomycin/spectinomycin resistance for bacteria selection.

The binary vector pZFN-nptII-70s::tDT were produced by following standard cloning procedures. Within the T-DNA of this vector, the cDNA of the Tdt sequence was cloned between the double CaMV 35S promoter and the nopaline synthase (NOS). The T-DNA also contains the neomycin phosphotransferase II (nptII) gene that confers resistance to a range of aminoglycoside antibiotics such as kanamycin or paromomycin and was used for the selection of transgenic plant cells and tissues. The NOS promoter and the pAG7 terminator flank the nptII gene The backbone of the binary vector contains the colE1 and the pVS1 origins for plasmid replication in Escherichia coli and Agrobacterium tumefaciens, respectively; and the aadA gene that confers streptomycin/spectinomycin resistance for bacteria selection.

The binary plasmid was transformed into AGL-1 Agrobacterium strain by a standard procedure.

2. Dex-Induced 35S-BnBBM-GR Enhances Shoot Formation from Beta vulgaris Calli in Hormone-Containing Tissue Culture Media

By applying the following method callus for Beta vulgaris (sugar beet) has been induced: Micropropagated shoots were used as starting material. Shoots were multiplied in medium containing MS salts, 30 g/l sucrose, 0.25 mg/l benzyladenine (BAP) and 10 g/l agar (pH 6.0). To induce friable callus, leaf explants were incubated for 7-8 weeks in media having MS salts, 15 g/l sucrose, 2 mg/l BAP, and 8 g/l agar (pH 6.0).

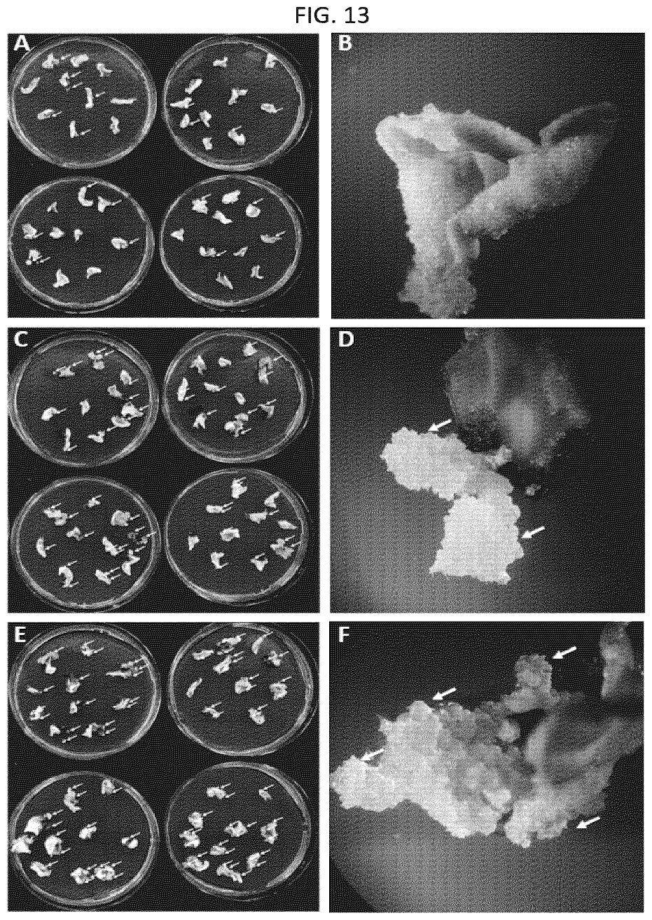

For Agrobacterium-mediated transformation friable calli were mounted in media containing MS salts, 30 g/l sucrose, 1 mg/l GA3, 1 mg/l TDZ, and 10 g/l agar (pH 6.0) and kept for 1 week in the dark at 24° C. Agrobacterium harbouring the vector of IPR232-pS-01 (FIG. 12) was grown in medium containing 5 g/l tryptone, 2.5 g/l yeast extract, 1 g/l NaCl, 5 g/l mannitol, 0.1 g/l MgSO₄×7H₂O, 0.25 g/l KH₂PO₄, 1 g/l glutamic acid (pH 7.0) supplemented with the appropriate antibiotics, at 28° C., for 24 h.

Calli were inoculated with Agrobacterium suspension at an OD600=0.3, the callus tissue and the Agrobacterium were incubated on a medium containing 440 mg/l CaCl₂×2H₂O, 170 mg/l KH₂PO₄, 1.9 g/l KNO₃, 180.7 mg/l MgSO₄, 1.65 g/l NH₄NO₃, 2 mg/l BAP, 40 pg/l acetosyringone, 20 g/l sucrose, 2 g/l glucose, and 10 g/l agar (pH 6.0) at 21° C. for 3 days in the dark.

Calli were subcultured to a medium containing MS salts (Duchefa #0222), 30 g/l sucrose, 1 mg/l GA3, 1 mg/l TDZ, 500 mg/l timentin, and 10 g/l agar (pH 6.0) and incubated in the dark, at 24° C. for 1 week.

To select the transgenic calli, samples were transferred to a medium containing MS salts (Duchefa #0222), 30 g/l sucrose, 1 mg/l GA3, 1 mg/l TDZ, 500 mg/l timentin, 10 g/l agar (pH 6.0), and 100 mg/l paromomycin. The calli were incubated at 24° C. in the light/dark cycle (16 h/8 h) for 3 weeks. Transgenic calli were selected and subcultured for several times in the same medium and conditions until the last selection that was done with and without addition of 1 μM dexamethasone (DEX) into the media for induction of BnBBM-GR protein.

The principle of the inducible expression of BnBBM is based on a fusion of the rat glucocorticoid receptor domain (GR) to the transcription factor BnBBM (BnBBM-GR). Upon induction with dexamethasone (DEX), the GR receptor domain will change conformation, so that the BnBBM-GR protein will enter the nucleus and generate the desired response.

Regenerating shoots were isolated and propagated in medium containing MS salts, 30 g/l sucrose, 0.25 mg/l benzyladenine (BAP), 100 mg/l kanamycin and 10 g/l agar (pH 6.0). Leaf explants were isolated from the green growing shoots for DNA extraction and PCR analysis, in order to confirm the presence of the transgene.

Selected shoots were rooted in medium containing MS salts, 30 g/l sucrose, 6.25 mg/l NAA, and 10 g/l agar (pH 6.0) and transferred to the green house for seed production.

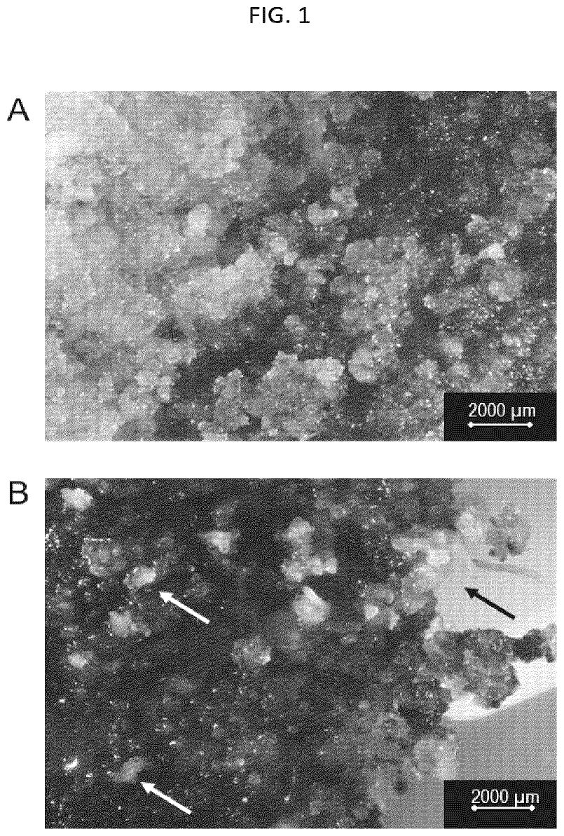

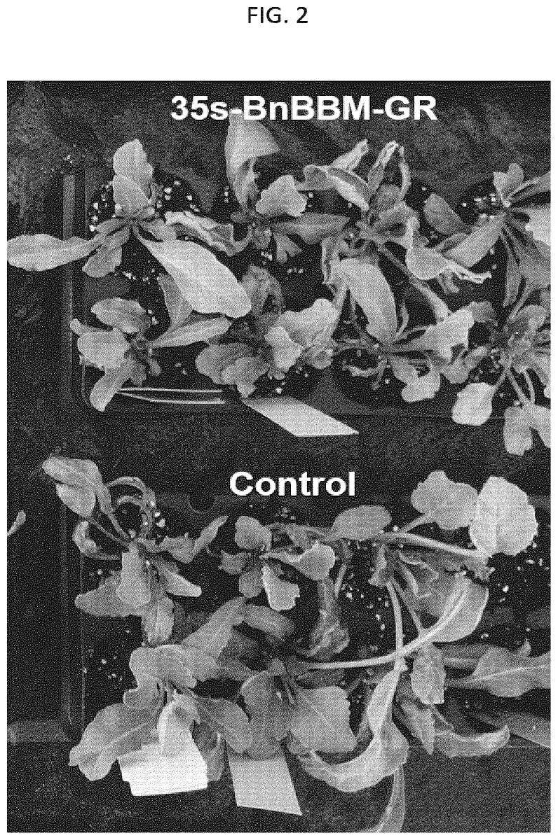

FIG. 1 shows that with DEX many shoots were formed from callus (B), while without DEX less or no shoot formation took place (A). Transgenic plants obtained from DEX-induced shoot formation show on soil similar morphology compared to non-transgenic wildtype plants (control) (see FIG. 2).

Thus, shoot formation from sugar beet callus is increased on DEX media in comparison to DEX free media The transgenic 35s-BnBBM-GR plants without inducer on soil do not show obvious phenotype.

3. BnBBM Dramatically Increases Callus-Based Transformation Efficiency in Beta vulgaris

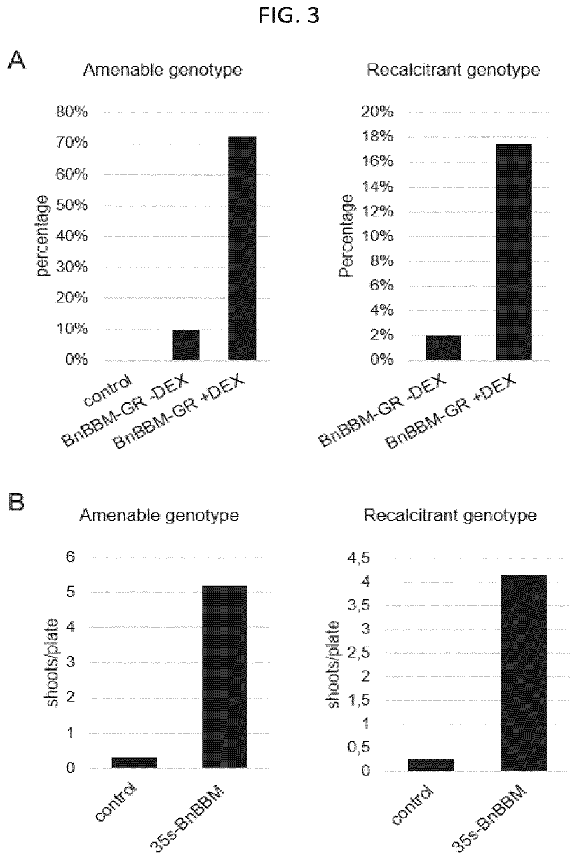

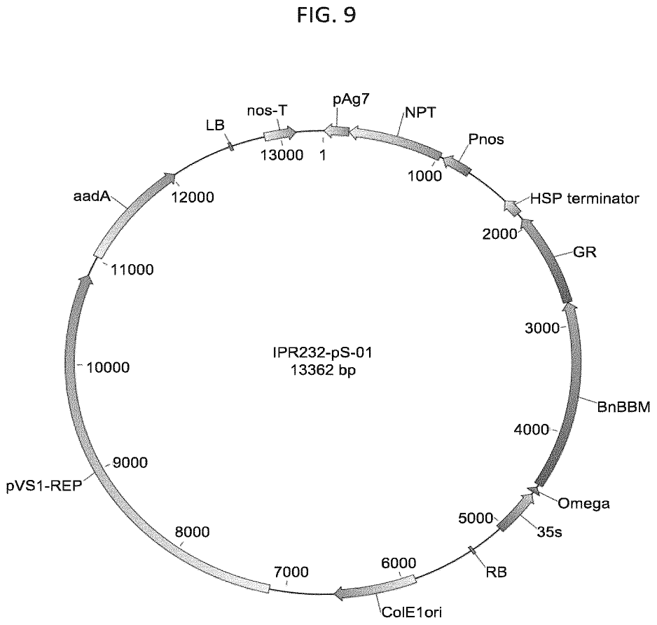

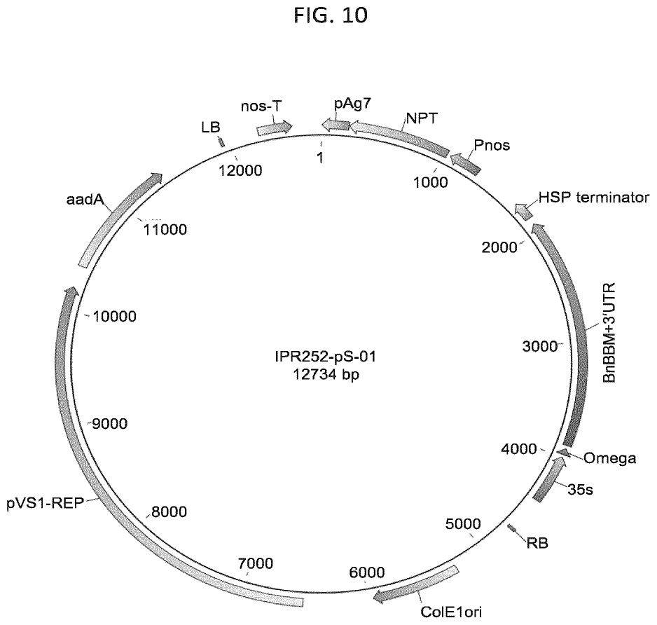

For quantification of transformation rate a) the inducible BnBBM system based on DEX/GR as described above as well as a constitutive overexpression of BnBBM have been tested on an amenable genotype and a recalcitrant genotype. As control served the respective genotype transformed with a vector carrying the fluorescent marker gene tdTomato.
a) The inducible BnBBM system was tested with and without addition of DEX to the media as described above. FIG. 3A shows the quantification of regenerated transgenic plants grown from induced shoots. It is apparent that by DEX-induction the frequency of transgenic plant formation is dramatically increased. In both genotypes a seven to eight fold increase in the transformation efficiency is observed, reaching of around 18% in the recalcitrant genotype and up to around 70% in the amenable genotype.
b) For the constitutive overexpression of BnBBM under the control of the CaMV 35S promoter the transformation efficiency is determined by counting transgenic plants obtained per plate (for detailed transformation protocol see Example 4). Generated transgenic plants are grown from shoots (derived from organogenesis) or embryos produced from callus (derived from embryogenesis) (FIG. 9). FIG. 3B shows a very similar tendency. The transformation efficiency of the constitutive expression BnBBM has been increased at least ten-fold for the amenable as well as for the recalcitrant genotype.

Thus, due to the combination of BnBBM-GR with DEX callus-based transformation efficiency is dramatically increased in amenable as well as recalcitrant genotypes. Similar results of breaking recalcitrance in sugar beet genotypes where also obtained with constitutive expression.

### 4. Constitutive (Over) Expression Gives Rise to Embryo and Seedlings

The same protocol for transformation as described in Example 2 has been used, except that the vector IPR252-pS-01 instead of the vector of IPR232-pS-01 has been used. As negative control, callus as described in Example 2 has been produced, but no agrobacterium infection took place. For shoot induction and propagation, the calli were harvested and transferred to the shoot induction media containing MS salts, 30 g/l sucrose, 1 mg/l GA3, 1 mg/l TDZ and 10 g/l agar (pH 6.0). The calli were incubated at 24° C. in the light/dark cycle (16 h/8 h) for 1-2 weeks.

Regenerated shoots were mounted and cultured in media containing MS salts, 30 g/l sucrose, 0.25 mg/l BAP, and 10 g/l agar (pH 6.0) and plants were grown at 24° C. in the light/dark cycle (16 h/8 h).

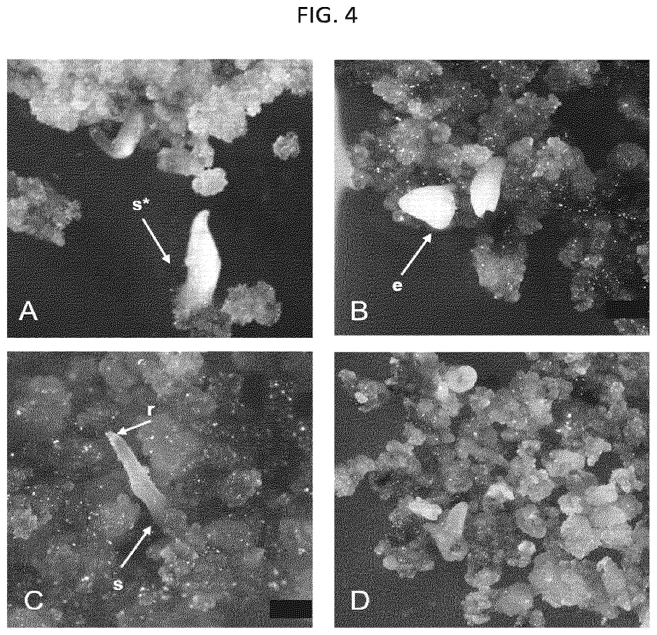

As for the inducible expression, even constitutive expression of BnBBM gives rise to embryo and seedling development (see FIGS. 4B and C) while standard callus-based transformation for sugar beet only generates shoots (see FIG. 4A). This shows that constitutive expression of BnBBM triggers embryogenesis and organogenesis. Many transgenic events could be produced compared to the control (FIG. 4 D).

Thus, constitutive and inducible expression of BnBBM in Beta vulgaris develop somatic embryos and shoots while standard callus-based transformation only generates shoots.

### 5. Constitutive (Over) Expression on Hormone-Free Medium

The same protocol as described in Example 4 has been used, except that the plant hormones TDZ and GA3 were not added to the media.

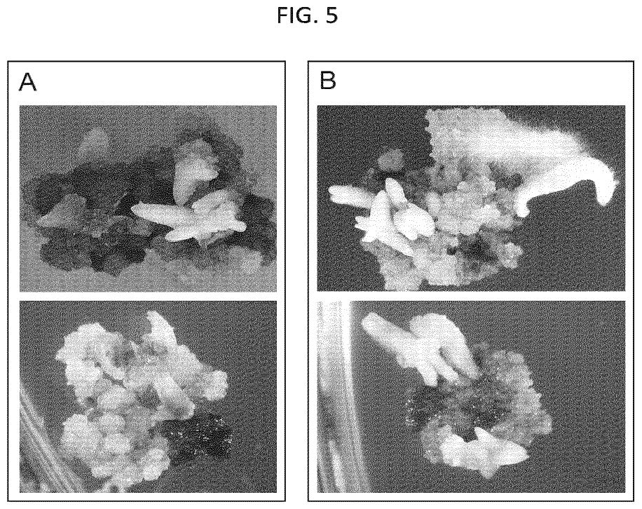

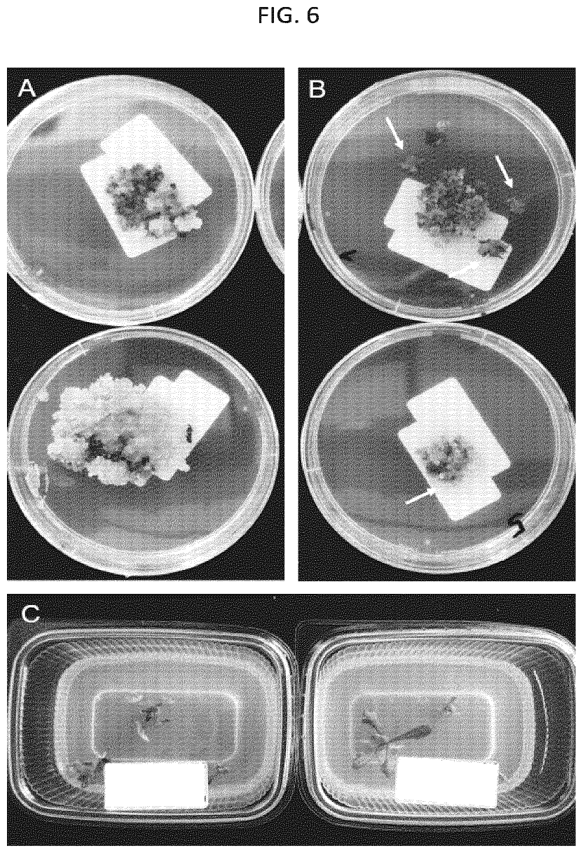

From callus tissue with a constitutive expression of BnBBM mainly embryos can be obtained on hormone free medium (FIG. 5B). When such tissue is placed on normal regeneration medium with hormones shoots and embryos are produced (FIG. 5A).

6. Co-Transformation with Inducible BnBBM

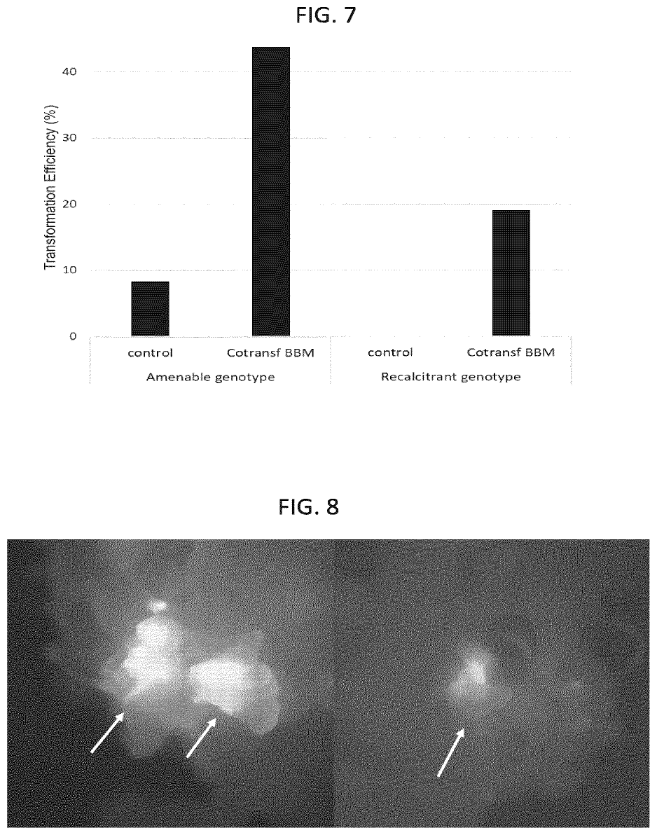

For co-transformation, agrobacteria harbouring each one of the vectors of interest were mixed 1:1 ratio (the vector 35s::BnBBM-GR and the vector 70s-tDT). Calli were inoculated with the obtained Agrobacterium suspension The callus tissue and the Agrobacterium were incubated at 21° C. for 3 days in the dark and subsequently subcultured as described in Example 2. Co-transformation shows significantly increased regenerative capacities of plant tissue leading to a more efficient co-transformation rate in amenable and recalcitrant genotypes (FIG. 7). Further, co-transformants were obtained three weeks earlier if BnBBM has been expressed.

Same result has been obtained for co-transformation with 35s BnBBM and the 70s-tDT vector.

The increased efficiency of co-transformation is of particular importance for the co-delivery of genome editing components for the targeted modification of the plant genome of co-transformed plant cells. By co-delivery of a CRISPR/Cpf1 system, SDN-1 events were successfully obtained after co-transformation with BnBBM and said genome editing components.

### 7. The Use of BnBBM and the Avoidance of Hormones Positively Affects The Development of the Tap Root (Storage Organ) at T0 Level

For Beta vulgaris and in particular for sugar beets or red beets, plants regenerated in vitro from callus show always an atypical phenotype particularly with respect to the hypocotyl and the roots. Typically, there is not a normal root body developed, so that the TO generation of e.g. sugar beets or red beets are not usable for testing root-related traits, e.g. sugar accumulation, nematode resistance, rhizomania resistance, etc or promoter expression analysis. Surprisingly, the inventors found that plant regenerated by means of BnBBM, inducible, but without hormones developed a normal tap root. Thereby, phenotyping of root-related traits and analysis of the storage-root ingredients are possible at TO level.

FIG. 12 shows T0 plants regenerated with inducible BnBBM and without hormone treatment.

### 8. Biolistic Delivery Method for Corn

With the microparticle bombardment, one or more booster genes, booster polypeptides, genome engineering components, and/or transgenes are co-delivered into immature embryos of corn via gold particles. Biolistic delivery has been conducted according the protocol as described in WO 2019/238911 A1

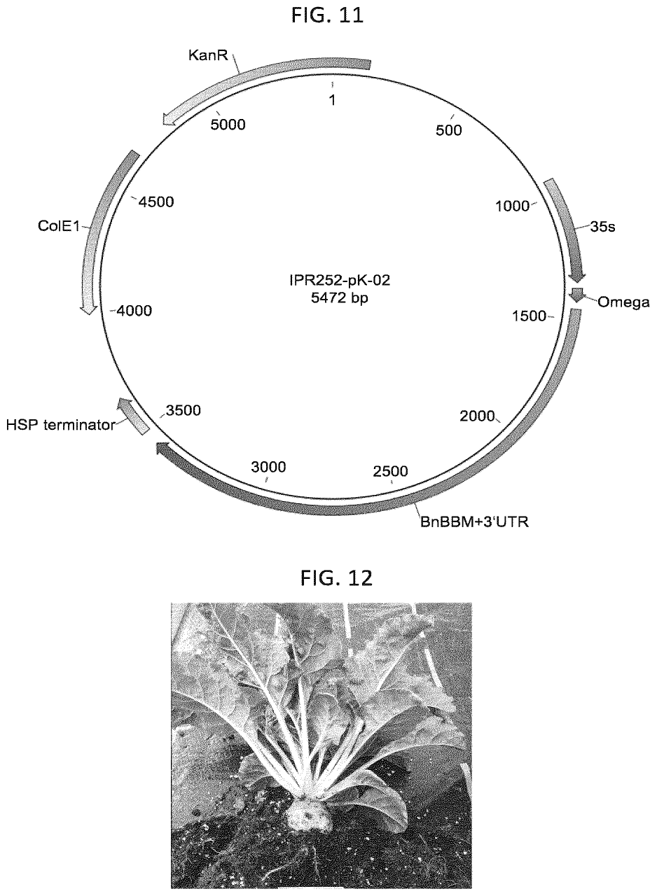

The booster genes, booster polypeptides, genome engineering components, and/or transgenes can be delivered into target cells using for instances a Bio-Rad PDS-1000/He particle gun or handheld Helios gene gun system. More than one construct can be co-delivered with genome engineering components into target cells simultaneously. For biolistic transformation of corn plants, the plasmid IPR252-pK-02 (FIG. 11) was used.

FIG. 8 shows the biolistic co-delivery of ZmWUS2, BnBBM and fluorescent marker Tdt. One month after bombardment three fluorescent embryonic structures form, which indicates that BnBBM combined with ZmWUS2 promotes maize regeneration from immature embryos.

### 9. SNP in Sugar Beet Medium Leads to Increased Callus Induction

Micropropagated shoots of different genotypes were used as starting material. The shoots were multiplied in medium containing MS salts, 30 g/l sucrose, 0.25 mg/l benzyladenine (BAP) and 10 g/l agar, pH 6.0.

To induce friable callus, leaf explants were incubated in medium containing MS salts, 15 g/l sucrose, 2 mg/l BAP, 8 g/l agar, 30 μM HP or 50 μM HP, pH 6.0 with 10 μM SNP, 100 μM SNP or without SNP, at 28° C. for 7-8 weeks.

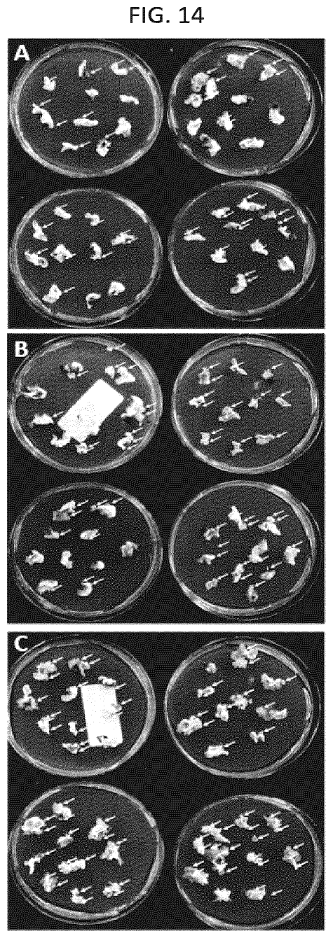

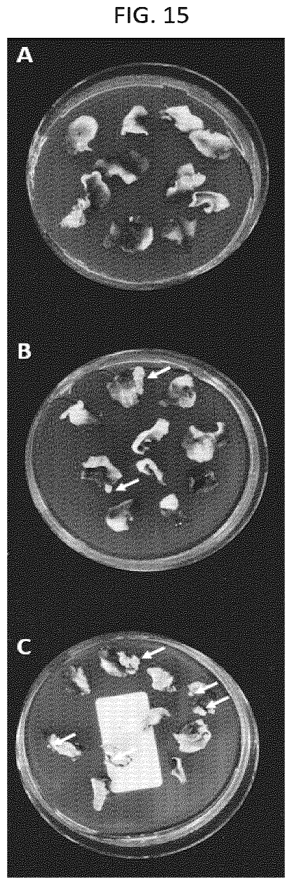

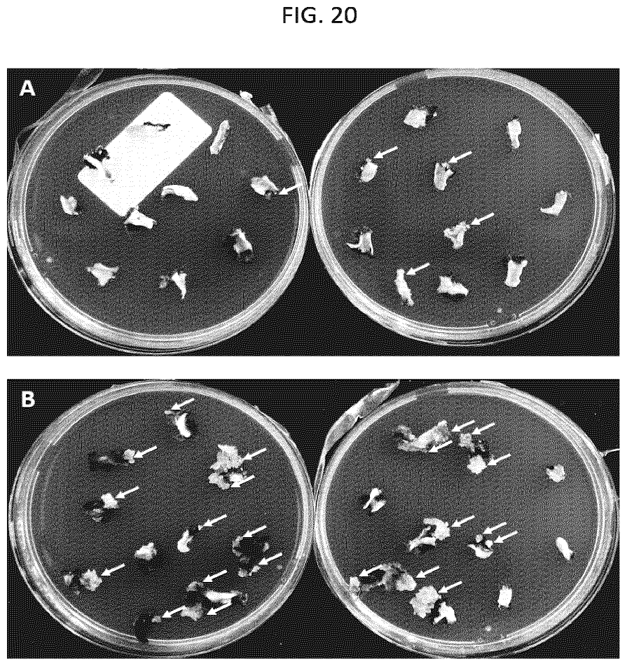

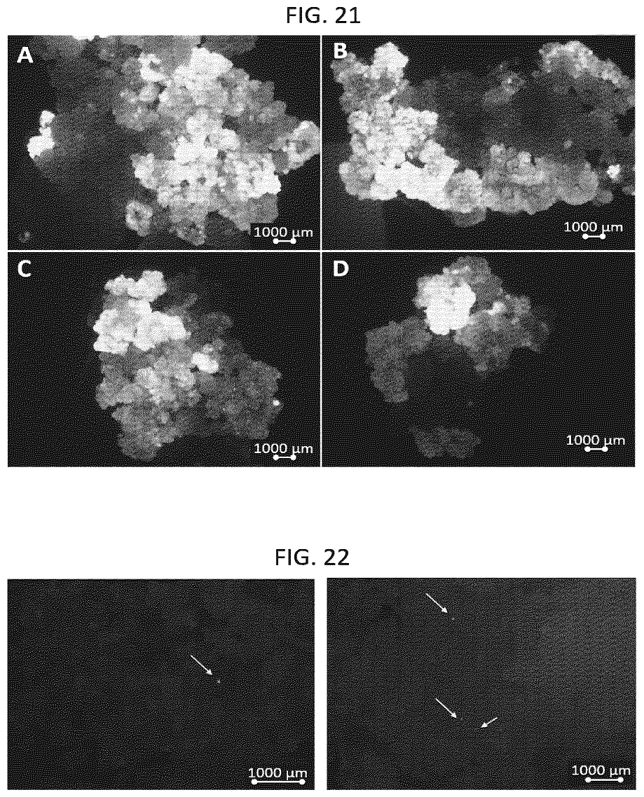

FIGS. 13-15 and 18-20 show that with SNP as media component callus tissue was induced from leaf explants of the genotypes 9BS0448, 1RV6183, 8RV6921,8RR3222, 6RR1798 and 7RV5706, while without SNP only less or no shoot formation was observable.

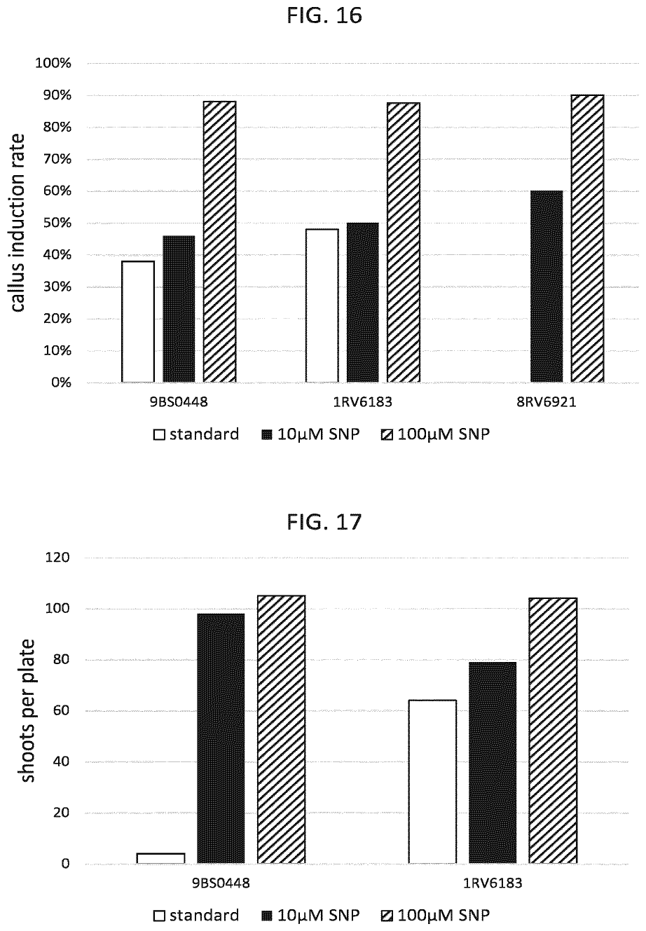

FIG. 16 shows the quantification of the callus induction rate from the genotypes 9BS0448, 1RV6183 and 8RV6921.

For the genotypes 9BS0448 and 1RV6183 only a minor effect of SNP is visible with 10 μM SNP in the medium in comparison to tissue cultivated on media without SNP, however when SNP is increased to 100 μM almost a doubling of the induction rate is obtained. For the recalcitrant genotype 8RV6921 the effect of SNP is even more drastic. Without SNP, no callus induction is observed, whereas with 10 μM SNP the callus induction rate was at a level of 60%, with 100 μM SNP the callus induction level were even at 90%,

### 10. SNP in Corn Medium Leads to Increased Callus Induction

immature embryos were used for the maize callus induction in the genotype 9D1473. Embryos of 0.5-1.4 mm were harvested 9-14 d after pollination.

The immature embryos were placed on the media containing MS salts, 3.3 mg/l Dicamba, 0.7 g/l Proline, 100 mg Casein hydrolysate, 100 mg/l Myo-Inositol, 30 g/l Sucrose, 95 mg/l L-Cysteine, 19.62 mg/l Acetosiringone, 15 mg/L AgNO₃, 2.3 g/l Gelrite, 1.25 mg/l CuSO₄, pH 5.8 at 25° C. for 7 days dark.

Subsequently the embryos were transferred to media containing MS salts, 3.3 mg/l Dicamba, 0.5 g/l MES, 100 mg/l Myo-Inositol, 30 g/l Sucrose, 0.7 g/l L-Proline, 100 mg/l Casein hydrolysate, 2.3 g/l Gelrite, 250 mg/l carbenicillin, 15 mg/l AgNO₃, pH 5.8 and subcultured every 2 weeks on C2 at 25° C. dark with different SNP regimes.

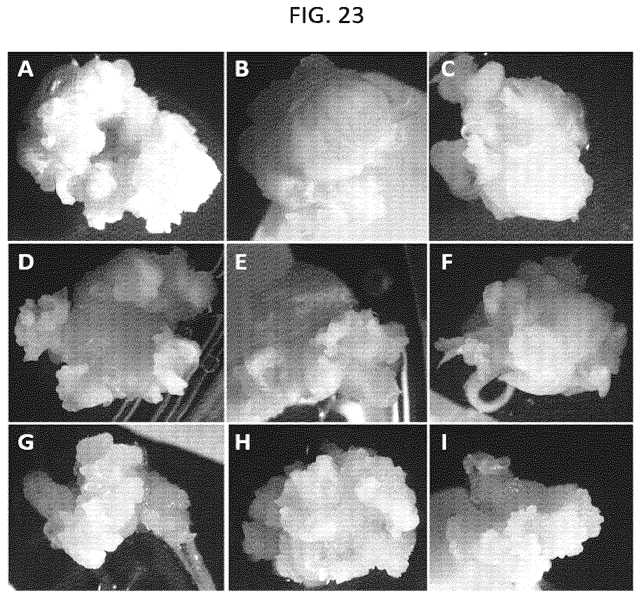

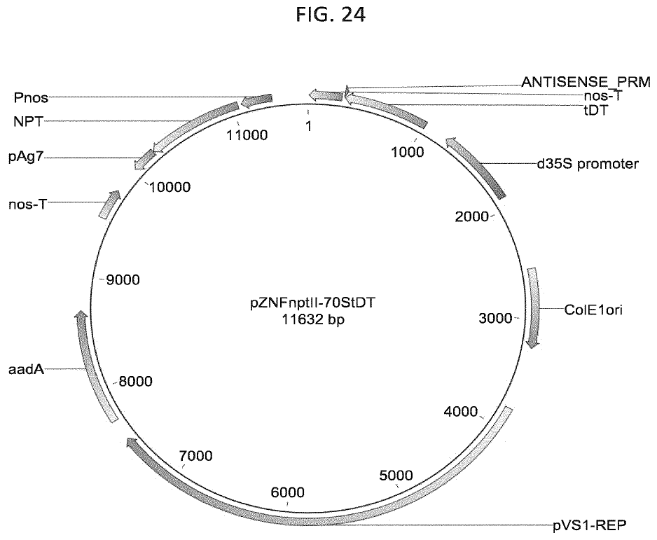

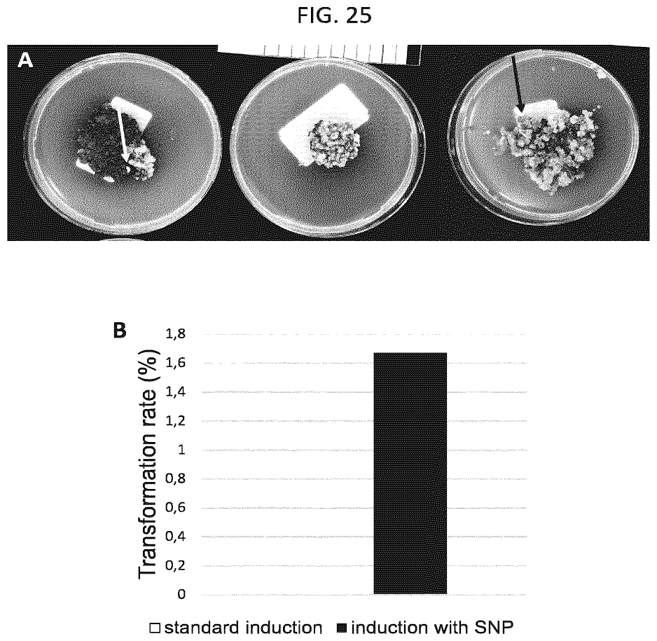

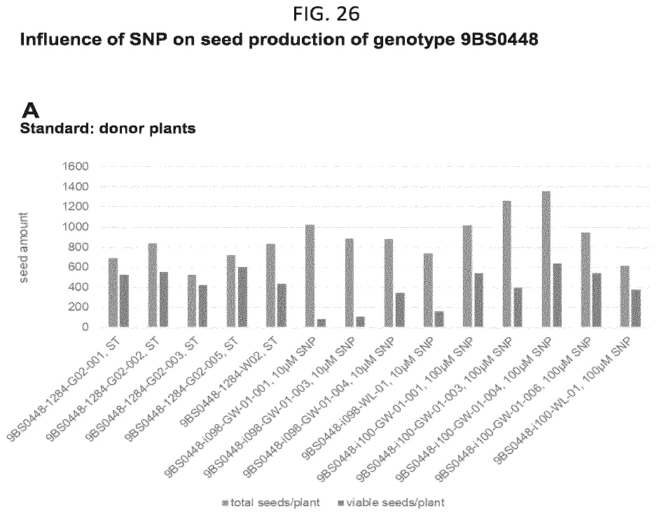

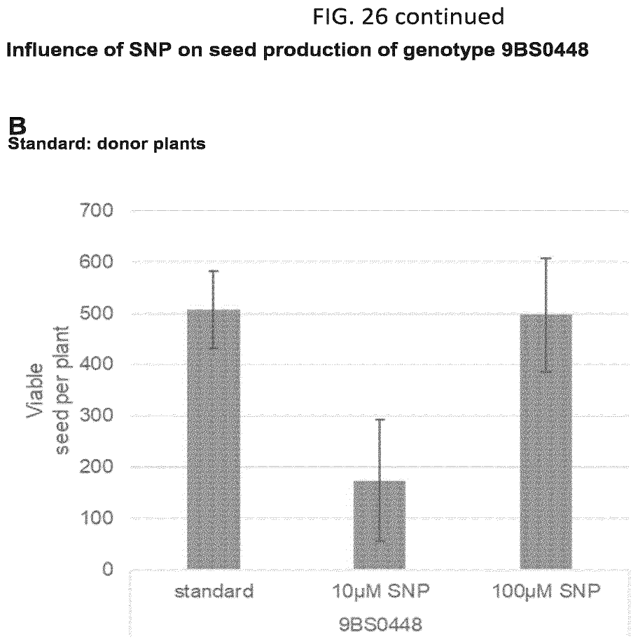

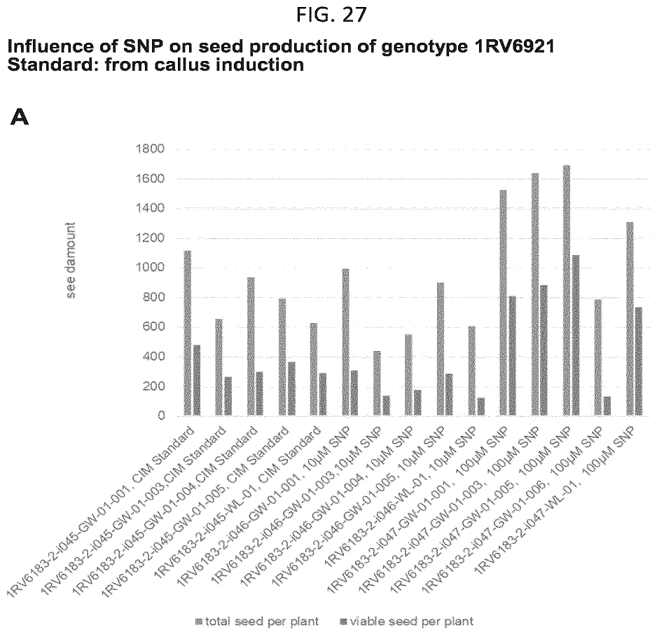

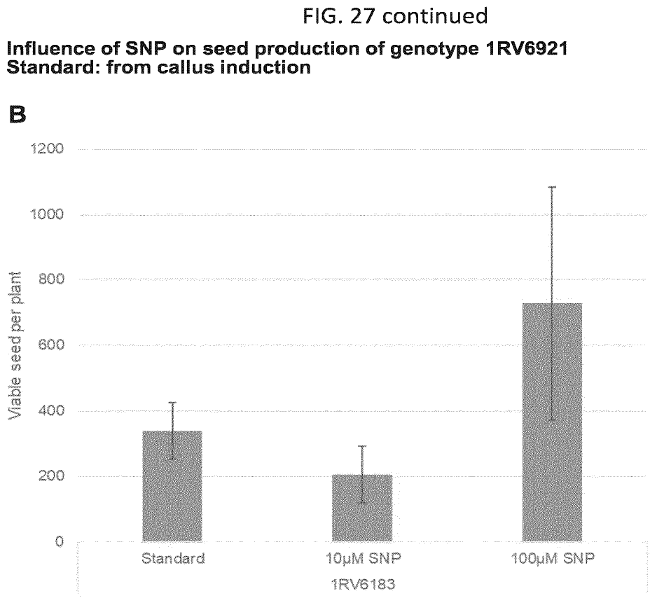

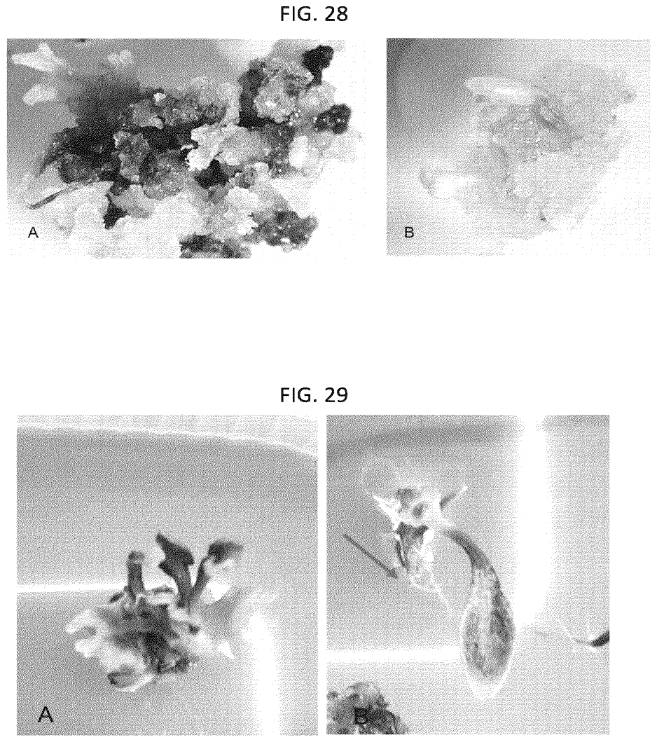

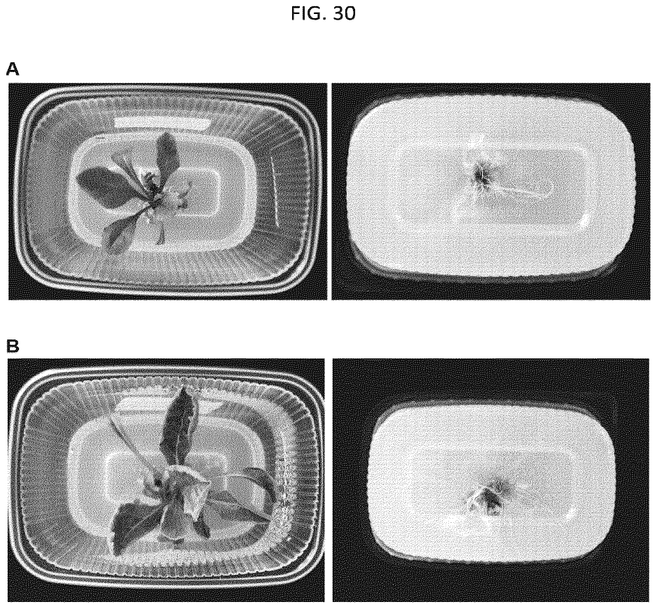

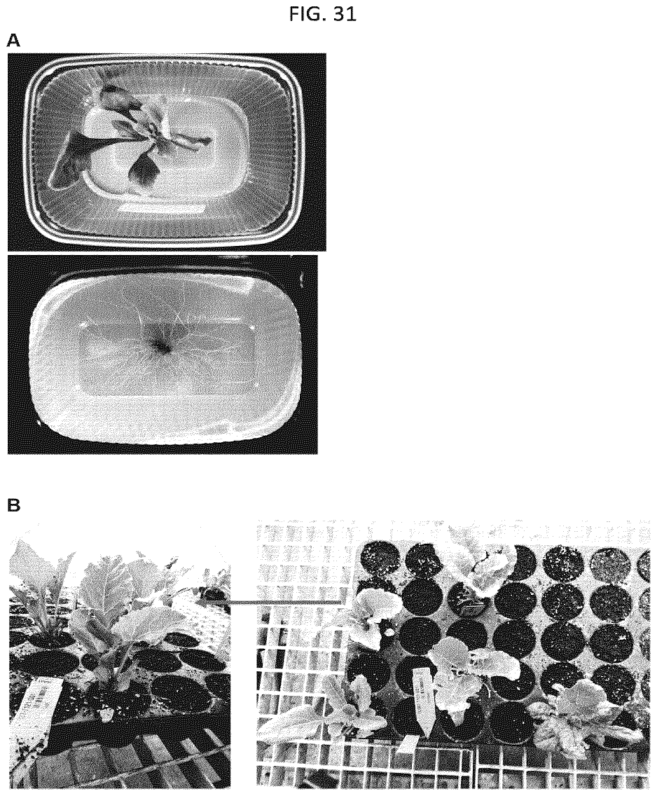

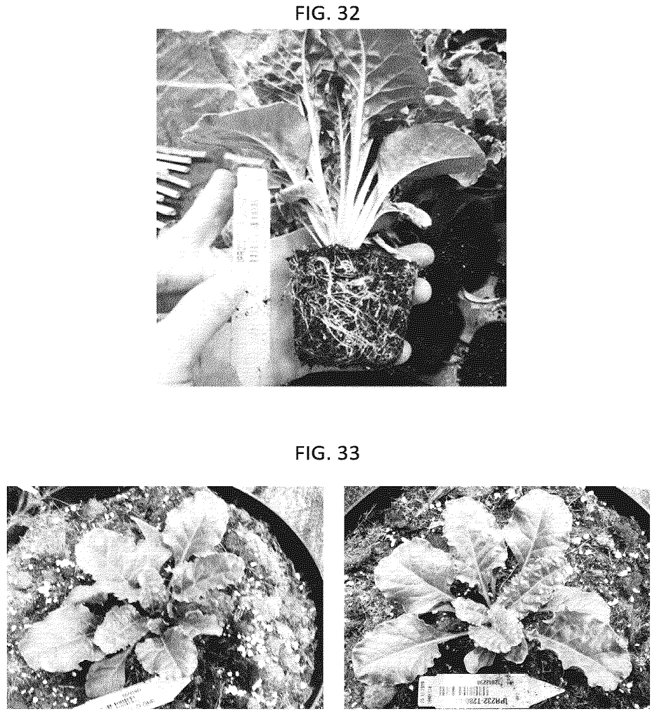

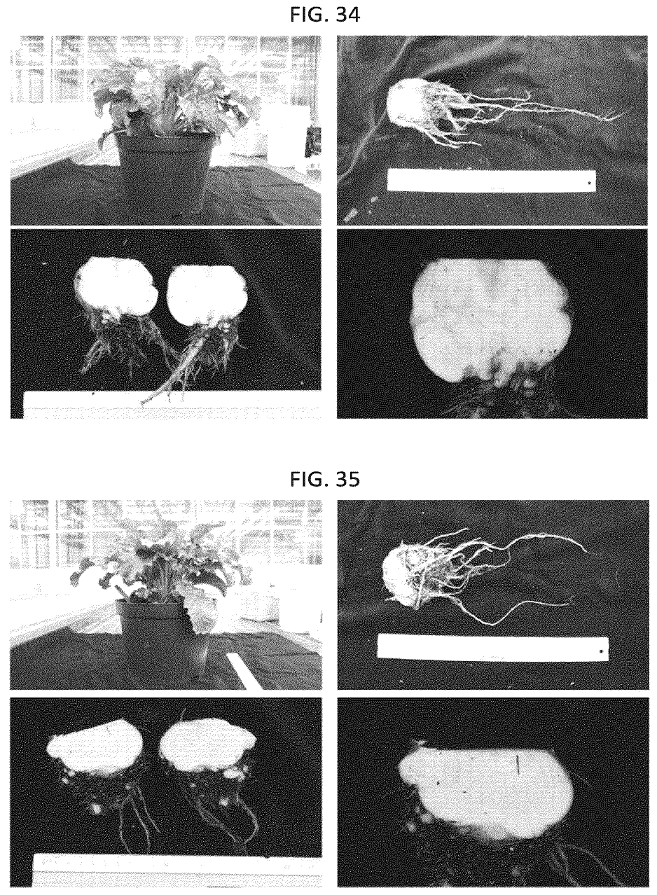

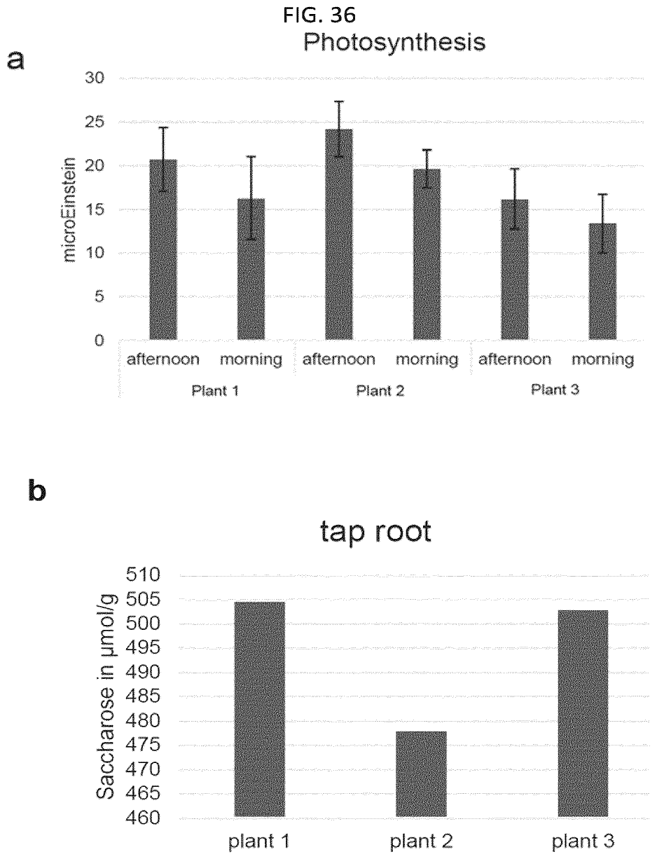

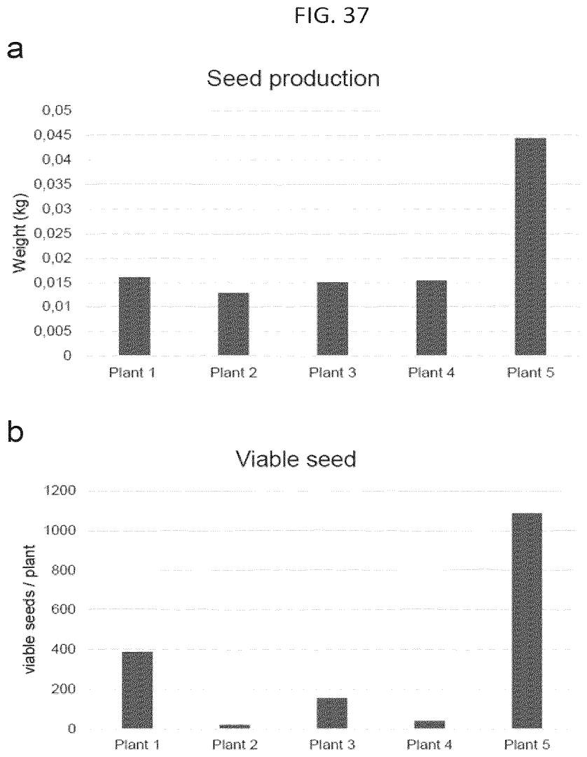

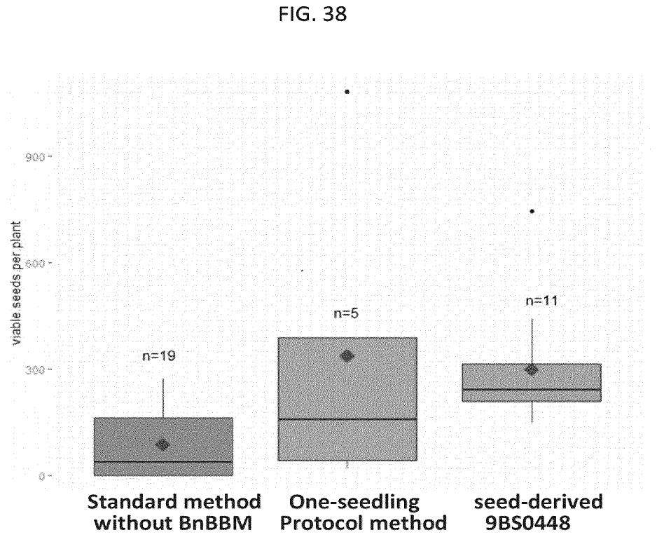

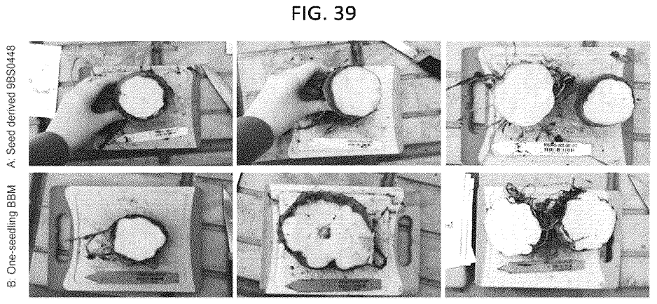

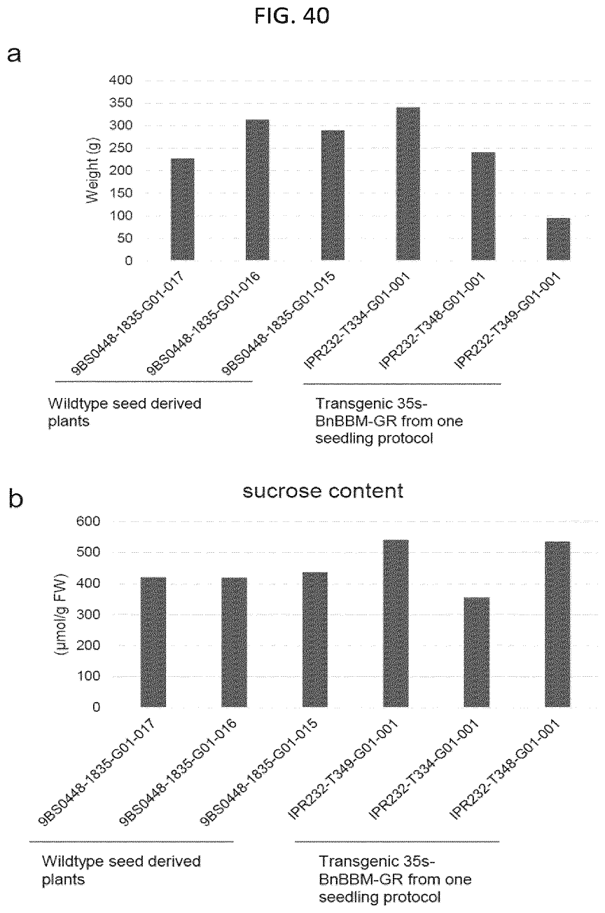

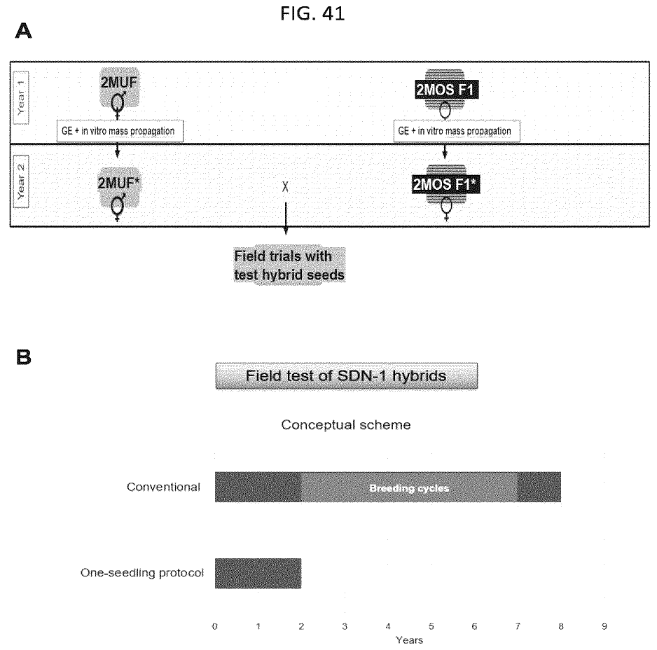

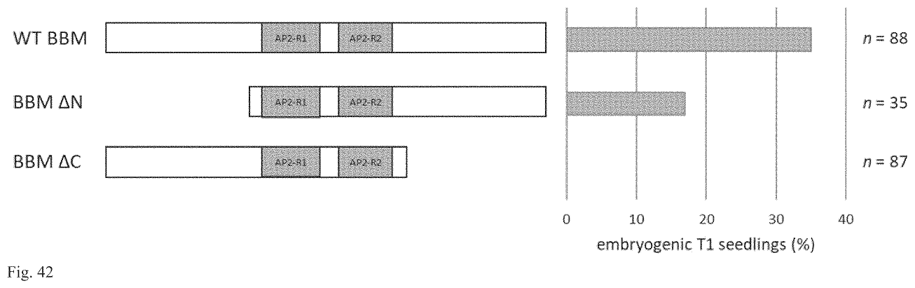

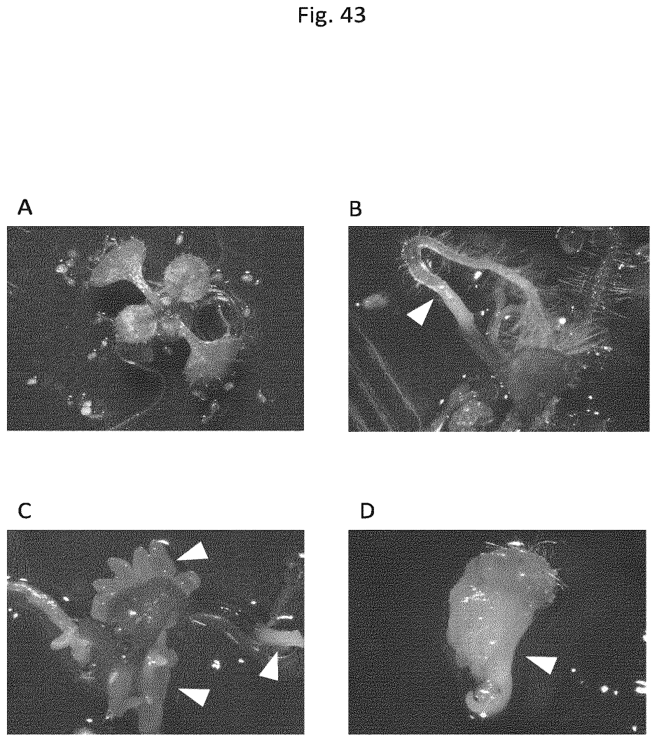

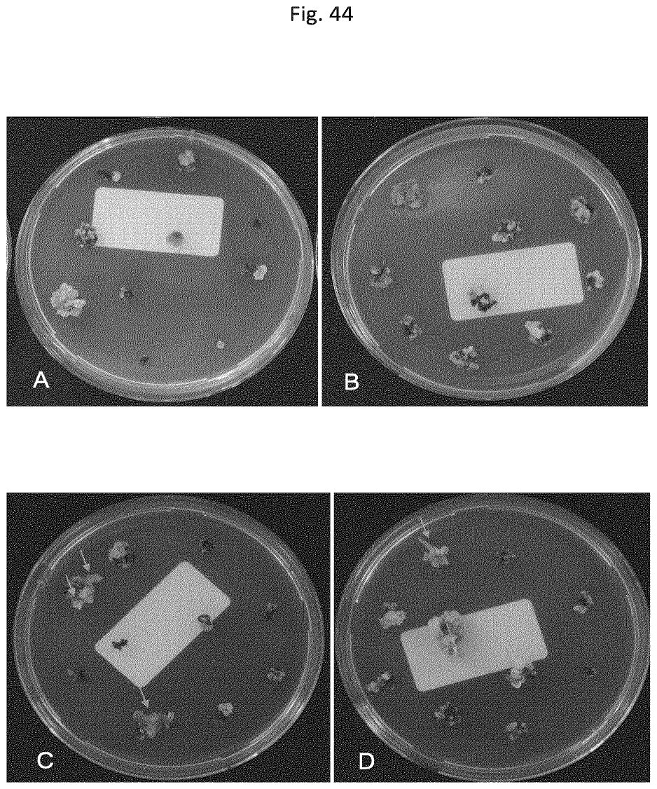

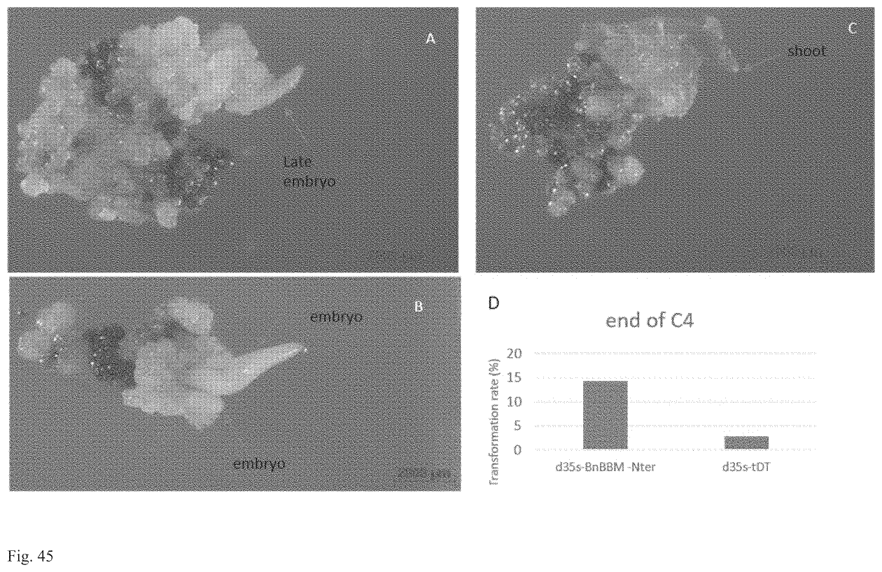

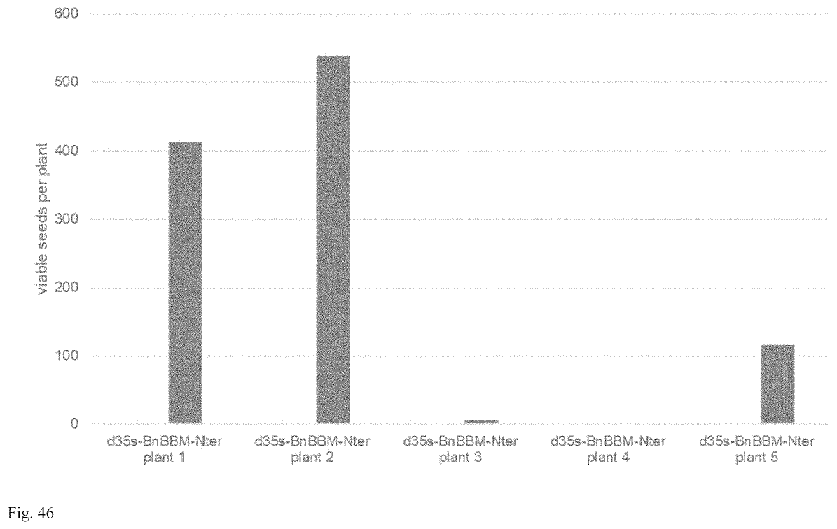

FIG. 23 shows for maize genotype 9D1437 that SNP as media component increased embryonic callus formation in comparison to embryos cultivated on media not containing SNP.

### 11. Sugar Beet Callus Induced by SNP can be Used for Agrobacterium Mediated Transformation

Sugar beet calli were induced as described above and friable calli were mounted in medium containing MS salts, 30 g/l sucrose, 1 mg/l GA3, 1 mg/l Thidiazuron (TDZ) and 10 g/l agar, pH 6.0 and kept for 1 week in the dark, 24° C.

Agrobacterium AGL-1 harbouring the vector pZFN-nptII-70s::tDT was grown in medium containing 5 g/l tryptone, 2.5 g/l yeast extract, 1 g/l NaCl, 5 g/l mannitol, 0.1 g/l MgSO₄×7H₂O, 0.25 g/l KH₂PO₄, 1 g/l glutamic acid, pH 7.0 supplemented with the appropriate antibiotics, at 28° C., for 24 h.

At an OD600 of 0.8, the callus tissue and the Agrobacterium were incubated in medium containing 440 mg/l CaCl₂×2H₂O, 170 mg/l KH₂PO₄, 1.9 g/l KNO₃, 180.7 mg/l MgSO₄, 1.65 g/l NH₄NO₃, 2 mg/l BAP, 40 pg/l Acetosyringone, 20 g/l sucrose, 2 g/l glucose, 10 g/l agar, pH 6.0, at 21° C. for 3 days in the dark.

Calli were subcultured to medium containing MS salts, 30 g/l sucrose, 1 mg/l GA3, 1 mg/l TDZ, 500 mg/l Timentin, 10 g/l agar, pH 6.0 and incubated in the dark, at 24° C. for 1 week.

To select the transgenic calli, samples were transferred to medium containing MS salts, 30 g/l sucrose, 1 mg/l GA3, 1 mg/l TDZ, 500 mg/l Timentin, 100 mg/l paromomycin, 10 g/l agar, pH 6.0 and incubated at 24° C. in the light/dark cycle (16 h/8 h) for 3 weeks.

Transgenic calli were selected and subcultured for several times in the same medium and conditions.

FIG. 21 shows that callus induced by SNP can be used for agrobacterium mediated transformation since a strong tdTomato signal was visible after the transformation with pZFN-nptII-70s::tDT. Therefore, callus induced by SNP can be potentially combined for example with genome editing approaches

### 12. Sugar Beet Callus Induced by SNP can be Used for Biolistic Delivery

Friable calli were produced as described above. An osmotic treatment was carried out by incubating the calli in medium containing MS salts, 30 g/l sucrose, 6.25 mg/l NAA, 10 g/l agar, pH 6.0 for 2.5 h at room temperature in the dark.

Preparation and DNA coating of the gold particles was done by standard procedures, as describe in the PDS-1000/He instruction manual. Gold particles were coated with pZFN-nptII-70s:tDT.

Calli were bombarded with a PDS-1000/He unit (Bio-Rad), using 30 ng gold particles coated with 500 ng DNA per shot. The rupture pressure was 450 psi and the target tissue was placed at 9 cm distance from the stopping screen.

FIG. 22 shows that callus induced by SNP can be used for biolistic transformation since tdTomato signals were visible after the bombardment with pZFN-nptII-70s::tDT. Therefore, callus induced by SNP can be potentially combined for example with genome editing approaches

### 13. Sugar Beet Shoot Regeneration

Friable calli were harvested from the induction and transferred to the shoot induction media containing MS salts, 30 g/il sucrose, 1 mg/l GA3, 1 mg/l Thidiazuron (TDZ), 10 g/il agar, pH 6.0. The calli were incubated at 24° C. in the light/dark cycle (16 h/8 h) for 1-2 weeks. Regenerated shoots were mounted and cultured in media containing MS salts, 30 g/l sucrose, 0.25 mg/l benzyladenine (BAP), 10 g/l agar, pH 6.0 and the plants are grown at 24° C. in the light/dark cycle (16 h/8 h). Shoots of the genotypes 9BS0448 and 1RV6183 regenerated from callus derived from SNP treatment does not show obvious phenotypes.

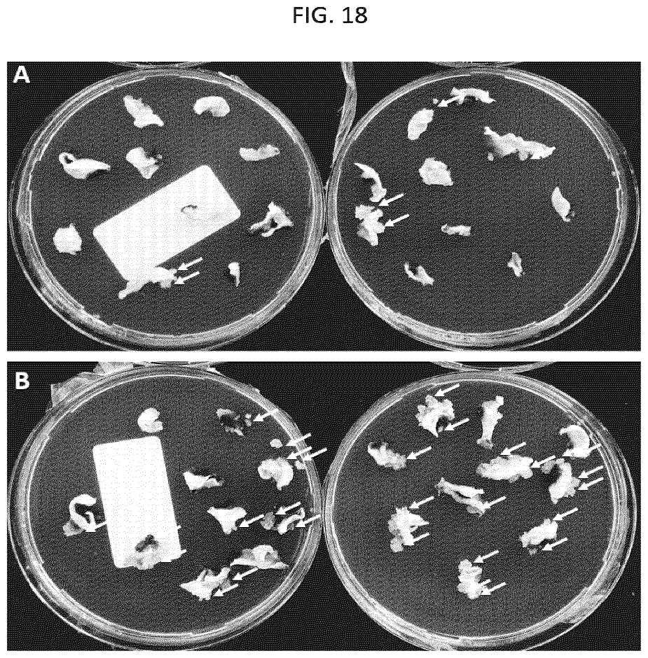

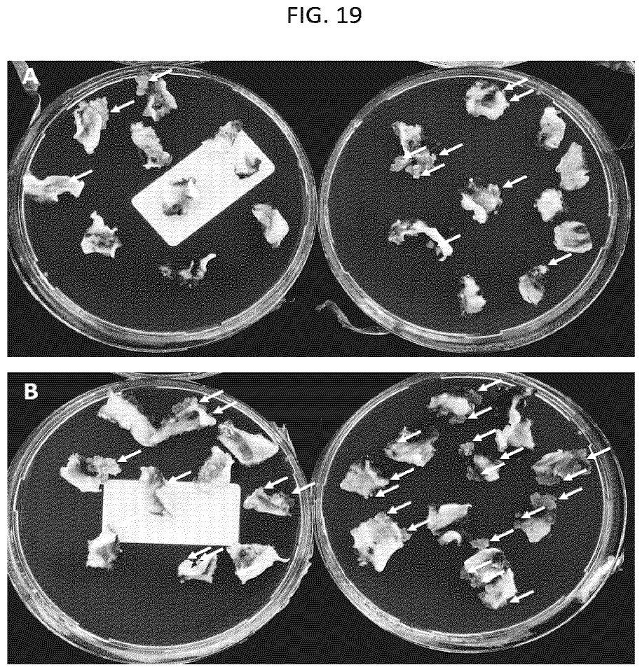

FIG. 17 shows that callus shoot induction of sugar beet genotypes 9BS0448 and 1 RV6183 was clearly increased when callus was induced with SNP.

For 9BS0448 only a very low number of shoots were obtained from callus without SNP treatment, whereas when callus of this genotype was induced with SNP a dramatic increase was observed.

With genotype 1 RV6183 significant number of shoots were obtainable from callus that received no SNP treatment, however also here a clear increase was detected when the callus was induced with SNP.

These results clearly show the beneficial influence of SNP treatment on shoot induction

14. Sugar Beet Callus of the Recalcitrant Genotype 1T4000 Induced by SNP and Transformed with BnBBM

Micropropagated shoots of the sugar beet genotype 1T4000 were induced as described above. To induce friable callus, leaf explants were incubated in medium described above with 100 μM SNP or without SNP, at 28° C. for 7-8 weeks.

The obtained sugar beet calli have been transformed by agrobacteria-mediated transformation or by biolistic delivery with BnBBM-GR under the control of a 70s promoter. No SNP was given to the media used during transformation.

FIG. 25 shows callus of the recalcitrant phenotype 1T4000 induced with SNP and transformed with BnBBM. With SNP a transformation rate of 1.67% was observed when transforming the tissue with BnBBM-GR under the control of a 70s promoter. Without SNP and BnBBM treatment, no shoots have been obtained.

### 15. Influence of SNP on Seed Production

The total number of seeds as well as the number of viable seeds were determined from sugar beet plants obtained by callus induction including SNP. The results were either compared to data from plants obtained by callus induction without SNP or to data from plants that have not gone through the cycle of callus induction and regeneration but were obtained conventionally (donor plants).

For these approaches, two sugar beet lines have been used (1RV6921 & 9BS0448) and two different SNP concentrations were applied in the callus induction media with 10 μM and with 100 μM.

FIG. 26 shows the results obtained with the sugar beet genotype 9BS0448, as comparison donor plants have been used that have not gone through the cycle of callus induction and regeneration but have been obtained conventionally. As shown in FIGS. 26 A and B treatment with a low SNP concentration (10 mμM) was leading to relatively low numbers of viable seeds per plant. The rate of viable seeds was approximately lowered by two thirds, when being compared to the donor plants as well as to plants which are obtained with a high SNP treatment for callus induction. Surprisingly, plants obtained from callus induction with a 10× higher SNP concentration produced as many viable seeds as the conventional donor plants.

This observation was unexpected, since plants obtained from callus induction and regeneration have normally a reduced seed production rate compared to conventional produced plants.

In FIG. 27 results regarding the number of viable seeds are shown from the sugar beet genotype 1 RV6921. As a control plants were used which had been generated from callus without SNP treatment. FIG. 27 b shows that the number of viable seeds per plant is again relatively low when the callus was induced with 10 μM SNP. Whereas callus induction with 100 μM lead to a clear increase in the number of viable seeds per plant, when being compared to plants from the 10 μM regime but also to plants were no SNP was used for callus induction. This observation supports the data of FIG. 26 and shows the beneficial effect of SNP treatment not only on the callus regeneration but also on the quality of the obtained plants.

16. Plant Development on Media with and without Hormones

Sugar beet calli were transformed with 35S-BnBBM-GR and placed either on standard medium with hormones (GA3 and BAP) or without hormones. By omitting the hormones GA3 and BAP as medium components somatic embryo formation was clearly more pronounced in comparison to calli cultivated on media with hormones (FIGS. 28 and B). In the standard protocol the development of shoots is clearly visible. In subsequent development, seedlings derived from embryos cultivated on hormone free media developed a normal root system (FIG. 29), whereas shoots, which were cultivated on media containing hormones, showed no roots (FIG. 29). This protocol for plant development is also called one-seedling protocol.

In FIGS. 30 and B plants are shown which have been developed from seedlings grown on hormone free media. These plants have no obvious phenotype and developed a normal root system without artificial induction of root growth.

It could be demonstrated that this beneficial effect on BnBBM is achievable by Agrobacterium transformation (see FIGS. 28-30) as well as by particle bombardment (FIG. 31). Transgenic 35S-BnBBM-GR plants obtained by transformation via particle bombardment developed in vitro also a proper root system (FIG. 31A). After transfer to the greenhouse these plants do not develop an obvious visible phenotype (FIG. 31B) and have a similar appearance like plants obtained by Agrobacterium mediated transformation.

17. Development of Plants after Transfer to Soil and Cultivation in the Greenhouse

Plants were transferred from hormone free media to soil after one to three months depending on the developmental status and were cultivated for one month in the greenhouse. In FIG. 32 the root system of such a Beta vulgaris plant is shown which has been developed from a seedling grown on hormone free media. The plant shows a robust root system and also the upper ground part of these plants have developed no obvious phenotype (FIG. 33).

After 4 months in the greenhouse the morphology of the tap root was evaluated. In the cross section, the overall size as well as the overall appearance no obvious abnormalities of the tap root morphology had been observed (FIGS. 34 and 35) Beside the tap root morphology also photosynthesis rate (FIG. 36a), and sucrose concentration in tap roots have been analysed (FIG. 36b).

As shown in FIG. 36a for three plants the photosynthesis rate has been determined two times a day. All results were approximately in the range of 15 microEinstein to 25 microEinstein.

In FIG. 36b the results of the sucrose content in the tap roots are shown in μmol/g for three different plants, the results are between 475 and 505 μmol/g.

### Starch and Soluble Sugar Contents

### Extraction

Plant material is extracted with 80% ethanol at 80° C. for 1h. Soluble sugars dissolve in ethanol. The extract is dried with help of a vacuum centrifuge and material resolved in water. The remaining extracted material will be ground if it had not already been so for ethanol extraction and again extracted with 0.2N KOH at 95° C. for 1h. Starch is partly hydrolyzed and dissolved at this step After cooling down the samples to room temperature, 1 M acetic acid is added to set the pH to 5-6. By addition of appropriate amounts of amyloglucosidase and alpha-amylase and incubation at 37° C. for at least 4 h and additionally at room temperature overnight starch is degraded to glucose.

### Determination of Concentrations:

Aqueous solutions are used to measure the concentrations of glucose, fructose, sucrose and starch (via glucose) by a coupled optical-enzymatic test using any type of photometer. The measuring buffer contains of 100 mM HEPES pH 7.4, 1 mM NAD, 2 mM ATP, 10 mM MgCl₂ and an appropriate amount of glucose-6-phosphate dehydrogenase. After addition of aqueous extract extinction/absorption is measured at a wavelength of I=340 nm (0 value). To measure glucose, an appropriate amount of hexokinase is added and measured long enough to convert all glucose to glucose-6-phosphate and fructose to fructose-6-phosphate. To measure fructose, an appropriate amount of phosphoglucoisomerase is added to convert all fructose-6-phosphate to glucose-6-phosphate. To measure sucrose, an appropriate amount of invertase (beta-fructosidase) is added to fully split sucrose into glucose and fructose which, in turn, are further converted to glucose-6-phosphate intermediary. Whenever in this series of enzyme reactions glucose-6-phosphate is produced, it is further oxidized to gluconate-6-phosphate with a simultaneous reduction of NAD to NADH. The latter reduction gives the read-out for the respective concentrations as NAD does not absorb light of 340 nm wavelength whereas NADH does. With help of the extinction coefficient of NADH at 340 nm and the Lambert-Beer quotation, the concentration of NADH in solution can be calculated. Knowing that glucose and fructose give rise to an equimolar concentration of NADH, both sugar concentrations can be calculated. For 1 mole of sucrose, 2 moles of NADH are formed. By also knowing the sample weight and volumes of liquids used in the process, the amounts of glucose, fructose, sucrose and starch (as mol hexose unit per mass) can be calculated form the concentrations measured.

The described data for plants derived from a hormone free transformation are in the normal range of seed derived plants.

The transgenic 35S-BnBBM-GR plants obtained by the hormone free transformation protocol show a normal development and a properly developed root system, furthermore these plants are also fertile as shown in FIG. 37. The seed weight in kg per plants has been determined from 5 different transgenic plants (FIG. 37), additionally the viable seeds per plants have been counted (FIG. 38).

The results for seed weight are around 0.015 kg/plant for 4 plants, one plant had almost 0.045 kg seeds. Viable seeds have been obtained from all 5 tested plants; however, the results show a certain range with 30 viable seeds per plant to 1100 viable seeds per plant.

In FIG. 38 the number of viable seeds per plants is compared between plants obtained by a standard Agrobacterium-based transformation without BnBBM, the described hormone free transformation method with BnBBM and plants derived from seeds of SB line 9BS0448 as reference. The presented results show that seed production from transgenic 35S-BnBBM-GR plants is better than from plants obtained by a standard Agrobacterium transformation method.

18. Analysis of Tap Roots from a Second Batch of Plants Derived in a Hormone Free Environment.

After 4 months of growth in the greenhouse tap roots were harvested from seed derived Beta vulgaris plants or from transgenic 35S-BnBBM-GR plants derived from Example 9. No obvious differences were identified as shown in FIGS. 39 and 40.

Furthermore, the tap root weight (FIG. 40) as well as the sucrose content (pmol/g FW (fresh weight)) (FIG. 40) were analysed. In FIG. 40a the root weight of WT (wild type) seed derived plants and transgenic 35S-BnBBM-GR from the hormone free approach are shown. Results vary between approx. 100-350 g For the sucrose content values between 350-520 μmol/g FW have been determined.

Overall, these results show that the tap root weight as well as the sucrose content of the transgenic plants from the described hormone free protocol are comparable with seed derived wild type controls.

### 19. Effect of Deletions at C- or N-Terminal of BnBBM

Experiments in Arabidopsis thaliana

### Vector Construction

The BBMΔ(1-161), and BBMΔ(390-579) deletion constructs used for stable transformation in Arabidopsis were PCR amplified and then cloned into the pTYG52 binary vector derived from pGSC1700 (Cornelissen and Vandewiele, 1989).

The respective sequences in Arabidopsis were under the control of a 35S promotor.

### Plant Transformation and Selection

All constructs were transformed using the floral dip method (Clough and Bent, 1998). Seeds were sterilized with liquid bleach as described previously (Horstman et al., 2017) and plated on ½MS-10 medium (half-strength Murashige and Skoog salts with vitamins, pH 5.8, 1% sucrose, and 0.8% agar) with phosphinothricin (PPT) for selection. T1-plants were cultured at 21° C. (16 h light/8 h dark cycle) for ten days, after which the phenotypes were assessed by visual inspection using a stereo microscope. T1-Seedlings were scored as non-embryogenic (FIG. 43A) or embryogenic (FIG. 43B-D).Typical phenotypes observed from transgenic seedlings expressing either the full-length BnBBM or the BBMΔ(1-161) construct are shown in FIG. 42.

The data suggest that the N-terminal region of the BnBBM protein is not absolutely required for its function, as such an N-terminal truncated protein is still able to induce somatic embryogenesis. By contrast, deletion of the C-terminal region of the BnBBM protein compromises its function, as its ectopic expression is not sufficient to induce somatic embryogenesis. The C-terminal protein sequences might be required for essential BBM protein-protein interactions, BBM transcriptional activation and/or BBM protein stability.

In sum, one can say there is no obvious explanation therefore this result could be understood as unexpected.

### Experiments in Sugar Beet

Sugar beet results were obtained for the full length and the N-truncated version of BnBBM in a similar way as described for Arabidopsis. A much higher transformation frequency using the N-truncated version of BnBBM can be achieved, compared to control experiments (FIG. 44 and FIG. 45D).

One can see a three-fold higher efficiency with the full length BnBBM version with regard to transformation frequency compared to the N-truncated version This is in line with the Arabidopsis data.

### Sequences:

Sequences:

| SEQ ID | | |
|---|---|---|
| NO: | Name | Description |
| 1 | BnBBM | cDNA of Brassica napus Babyboom |
| 2 | BnBBM | protein of Brassica napus Babyboom |
| 3 | BnBBM + 3′UTR | DNA sequence of Brassica napus Babyboom with |
| | | 3′UTR |
| 4 | BnBBM + GR | DNA sequence of Brassica napus Babyboom with |
| | | rat glucocorticoid receptor domain |
| 5 | pZFNnptII-70s tDT | Vector sequence |
| 6 | NPT | Neomycin phosphotransferase |
| 7 | tDt | tdTomato, orange fluorescent protein |
| 8 | d35S | double 35S promoter |
| 9 | ColE1 ori | Col E1 origin of replication |
| 10 | pVS1 REP | pVS1 Replicon |
| 11 | aadA | Aminoglycoside 3′-adenyl transferase from |
| | | Escherichia coli |
| 12 | ZmWUS2 | cDNA sequence of ZmWUS2 |
| 13 | ZmWUS2 | protein sequence of ZmWUS2 |
| 14 | IPR232-pS-01 | Full length vector |
| 15 | IPR252-pS-01 | Full length vector |
| 16 | IPR 252-pK-02 | Full length vector |
| 17 | BnBBM cDNA Ndel | cDNA BnBBM Version with shortened N terminus |
| 18 | BnBBM cDNA Cdel | cDNA BnBBM Version with shortened C terminus |
| 19 | BnBBM PRT Ndel | PRT BnBBM Version with shortened N terminus |
| 20 | BnBBM PRT Cdel | PRT BnBBM Version with shortened C terminus |

1-15. (canceled)

16. A method of inducing shoot formation from at least one plant cell, the method comprising:
(a) incubating the at least one plant cell in the presence of sodium nitroprusside (SNP);
(b) introducing into the at least one plant cell to be used in step (a) or into at least one cell of the callus obtained in step (a) an expression cassette comprising a coding nucleotide sequence selected from
(i) the nucleotide sequence of SEQ ID NO: 1; and
(ii) a nucleotide sequence encoding a polypeptide having the amino acid sequence of SEQ ID NO: 2,
wherein the nucleotide sequence is operably linked to a heterologous constitutive regulatory element or a heterologous inducible regulatory element; and
(c) cultivating the callus obtained in step (b) under conditions promoting growth of shoots out of the callus, wherein in the callus the polypeptide is expressed from the expression cassette constitutively or upon induction of the heterologous inducible regulatory element.

17. The method of claim 16, wherein the heterologous constitutive regulatory element is a constitutive promoter or the heterologous inducible regulatory element is an inducible promoter or an inducible expression system.

18. The method of claim 16, wherein the at least one plant cell of step (a) is a somatic or embryonic cell.

19. The method of claim 16, wherein the at least one plant cell is a plant cell from Beta vulgaris, Zea mays or Spinacia oleracea.

20. The method of claim 16, wherein in step (c), shoots are directly grown from the callus.

21. The method of claim 16, further comprising a step of
(d1) introducing at least one nucleotide sequence of interest into the at least one plant cell or a predecessor thereof to be used in step (a), into at least one cell of the callus obtained in step (a) which itself or a progeny thereof is then to be used in step (b) or has been used in step (b), and/or
(d2) modifying the genome of the at least one plant cell or a predecessor thereof to be used in step (a), of the at least one cell of the callus obtained in step (a) which itself or a progeny thereof is then to be used in step (b) or has been used in step (b), by introducing into said cell a single stranded DNA break (SSB) inducing enzyme or a double stranded DNA break (DSB) inducing enzyme which recognizes a predetermined site in the genome of said cell, and optionally a repair nucleic acid molecule, or a single stranded DNA break (SSB) inducing enzyme which recognizes a predetermined site in the genome of said cell and is fused to a base editor enzyme,
wherein the modification of said genome is selected from
I) a replacement of at least one nucleotide;
II) a deletion of at least one nucleotide;
III) an insertion of at least one nucleotide; and
IV) any combination of I)-III).

22. A method of increasing plant regeneration from at least one plant cell, the method comprising:
(a) incubating the at least one plant cell in the presence of sodium nitroprusside (SNP);
(b) introducing into the at least one plant cell to be used in step (a) or into at least one cell of the callus obtained in step (a) an expression cassette comprising a coding nucleotide sequence selected from
(i) the nucleotide sequence of SEQ ID NO: 1; and
(ii) a nucleotide sequence encoding a polypeptide having the amino acid sequence of SEQ ID NO: 2,
wherein the nucleotide sequence is operably linked to a heterologous constitutive regulatory element or a heterologous inducible regulatory element; and
(c) cultivating the callus obtained in step (b) under conditions promoting growth of embryos and/or shoots out of the callus, wherein in the callus the polypeptide is expressed from the expression cassette constitutively or upon induction of the heterologous inducible regulatory element,
wherein the plant regeneration rate is increased relative to a plant cell that has not been incubated in the presence of SNP and/or has not had the expression cassette of step (b) introduced.

23. The method of claim 22, wherein the heterologous constitutive regulatory element is a constitutive promoter or the heterologous inducible regulatory element is an inducible promoter or an inducible expression system.

24. The method of claim 22, wherein the at least one plant cell of step (a) is a somatic or embryonic cell.

25. The method of claim 22, wherein the at least one plant cell is a plant cell from Beta vulgaris, Zea mays or Spinacia oleracea.

26. The method of claim 22, wherein in step (c), embryos or shoots are directly grown from the callus, and optionally the cultivating is carried out in a medium free of plant hormones.

27. The method of claim 22, further comprising a step of
(d1) introducing at least one nucleotide sequence of interest into the at least one plant cell or a predecessor thereof to be used in step (a), into at least one cell of the callus obtained in step (a) which itself or a progeny thereof is then to be used in step (b) or has been used in step (b), and/or
(d2) modifying the genome of the at least one plant cell or a predecessor thereof to be used in step (a), of the at least one cell of the callus obtained in step (a) which itself or a progeny thereof is then to be used in step (b) or has been used in step (b), by introducing into said cell a single stranded DNA break (SSB) inducing enzyme or a double stranded DNA break (DSB) inducing enzyme which recognizes a predetermined site in the genome of said cell, and optionally a repair nucleic acid molecule, or a single stranded DNA break (SSB) inducing enzyme which recognizes a predetermined site in the genome of said cell and is fused to a base editor enzyme,
wherein the modification of said genome is selected from
I) a replacement of at least one nucleotide;
II) a deletion of at least one nucleotide;
III) an insertion of at least one nucleotide; and
IV) any combination of I)-III).

28. A method of producing a plant from at least one plant cell, the method comprising:
(a) incubating the at least one plant cell in the presence of sodium nitroprusside (SNP);
(b) introducing into the at least one plant cell to be used in step (a) or into at least one cell of the callus obtained in step (a) an expression cassette comprising a coding nucleotide sequence selected from
(i) the nucleotide sequence of SEQ ID NO: 1; and
(ii) a nucleotide sequence encoding a polypeptide having the amino acid sequence of SEQ ID NO: 2,
wherein the nucleotide sequence is operably linked to a heterologous constitutive regulatory element or a heterologous inducible regulatory element;
(c) cultivating the callus obtained in step (b) under conditions promoting growth of embryos and/or shoots out of the callus, wherein in the callus the polypeptide is expressed from the expression cassette constitutively or upon induction of the heterologous inducible regulatory element; and
(d) regenerating a plant from the embryo and/or shoot resulting from step (c).

29. The method of claim 28, wherein the heterologous constitutive regulatory element is a constitutive promoter or the heterologous inducible regulatory element is an inducible promoter or an inducible expression system.

30. The method of claim 28, wherein the at least one plant cell of step (a) is a somatic or embryonic cell.

31. The method of claim 28, wherein the at least one plant cell is a plant cell from Beta vulgaris, Zea mays or Spinacia oleracea.

32. The method of claim 28, wherein in step (c), embryos or shoots are directly grown from the callus, and optionally the cultivating is carried out in a medium free of plant hormones.

33. The method of claim 28, further comprising a step of
(d1) introducing at least one nucleotide sequence of interest into the at least one plant cell or a predecessor thereof to be used in step (a), into at least one cell of the callus obtained in step (a) which itself or a progeny thereof is then to be used in step (b) or has been used in step (b), and/or
(d2) modifying the genome of the at least one plant cell or a predecessor thereof to be used in step (a), of the at least one cell of the callus obtained in step (a) which itself or a progeny thereof is then to be used in step (b) or has been used in step (b), by introducing into said cell a single stranded DNA break (SSB) inducing enzyme or a double stranded DNA break (DSB) inducing enzyme which recognizes a predetermined site in the genome of said cell, and optionally a repair nucleic acid molecule, or a single stranded DNA break (SSB) inducing enzyme which recognizes a predetermined site in the genome of said cell and is fused to a base editor enzyme,
wherein the modification of said genome is selected from
I) a replacement of at least one nucleotide;
II) a deletion of at least one nucleotide;
III) an insertion of at least one nucleotide; and
IV) any combination of I)-III).

34. A method of promoting somatic embryogenesis or organogenesis of a plant, comprising the following steps:
(a) inducing callus formation from at least one plant cell comprising incubating the at least one plant cell in the presence of sodium nitroprusside (SNP), and
(b) introducing into the at least one plant cell to be used in step (a) or into at least one cell of the callus obtained in step (a) an expression cassette comprising a nucleotide sequence selected from:
(i) the nucleotide sequence of SEQ ID NO: 1; and
(ii) a nucleotide sequence encoding a polypeptide having the amino acid sequence of SEQ ID NO: 2,
wherein the nucleotide sequence is operably linked to a first heterologous constitutive regulatory element or a first heterologous inducible regulatory element;
(c) cultivating the callus obtained in step (b) under conditions promoting growth of embryos and/or shoots out of the callus, wherein in the callus the polypeptide is expressed from the expression cassette constitutively or upon induction of the heterologous inducible regulatory element; and
(d) introducing into the at least one plant cell
(I) the expression cassette further comprising a second nucleotide sequence selected from:
(i) the nucleotide sequence of SEQ ID NO: 12, and
(ii) a nucleotide sequence encoding a polypeptide having the amino acid sequence of SEQ ID NO: 13,
wherein the second nucleotide sequence is operably linked to the first heterologous constitutive regulatory element or the first heterologous inducible regulatory element or to a second heterologous constitutive regulatory element or a second heterologous inducible regulatory element; or
(II) an mRNA encoding a polypeptide having the amino acid sequence of SEQ ID NO: 13; or
(III) a polypeptide having the amino acid sequence of SEQ ID NO: 13; or
(IV) inducing in said at least one plant cell an enhanced expression level of an endogenous gene encoding the polypeptide having the amino acid sequence of SEQ ID NO: 13, and
wherein cultivating the callus in step (c) to promote growth of embryos and/or shoots out of the callus is carried out under conditions wherein in the callus the polypeptide having the amino acid sequence of SEQ ID NO: 2 is expressed from the expression cassette, and the polypeptide having the amino acid sequence of SEQ ID NO: 13 is expressed from the expression cassette, is translated from introduced mRNA, is expressed at an enhanced level from the endogenous gene, or is present.

35. The method of claim 34, wherein:
(i) the heterologous constitutive regulatory element is a constitutive promoter or the heterologous inducible regulatory element is an inducible promoter or an inducible expression system,
(ii) the at least one plant cell of step (a) is a somatic or embryonic cell,
(iii) the at least one plant cell is a plant cell from Beta vulgaris, Zea mays or Spinacia oleracea, and/or
(iv) in step (c), embryos or shoots are directly grown from the callus, and optionally the cultivating is carried out in a medium free of plant hormones

36. The method of claim 35, further comprising a step of
(d1) introducing at least one nucleotide sequence of interest into the at least one plant cell or a predecessor thereof to be used in step (a), into at least one cell of the callus obtained in step (a) which itself or a progeny thereof is then to be used in step (b) or has been used in step (b), and/or
(d2) modifying the genome of the at least one plant cell or a predecessor thereof to be used in step (a), of the at least one cell of the callus obtained in step (a) which itself or a progeny thereof is then to be used in step (b) or has been used in step (b), by introducing into said cell a single stranded DNA break (SSB) inducing enzyme or a double stranded DNA break (DSB) inducing enzyme which recognizes a predetermined site in the genome of said cell, and optionally a repair nucleic acid molecule, or a single stranded DNA break (SSB) inducing enzyme which recognizes a predetermined site in the genome of said cell and is fused to a base editor enzyme,
wherein the modification of said genome is selected from
I) a replacement of at least one nucleotide;
II) a deletion of at least one nucleotide;
III) an insertion of at least one nucleotide; and
IV) any combination of I)-III).
